(19) United States
(12) Patent Application Publication
(10) Pub. No.: US 2009/0312335 A1
(43) Pub. Date: Dec. 17, 2009

(54) Hiv Integrase Inhibitors

(76) Inventors: John S Wai, Harleysville, PA (US); Peter D. Williams, Harleysville, PA (US); Terry A. Lyle, Lederach, PA (US)

Correspondence Address: MERCK AND CO., INC, P O BOX 2000, RAHWAY, NJ, 07065-0907, US

(21) Appl. No.: 12/084,158

(22) PCT Filed: Oct. 23, 2006
(86) PCT No.: PCT/US2006/041280

§ 371 Date: Apr. 25, 2008

Related U.S. Application Data
(60) Provisional application No. 60/730,666, filed on Oct. 27, 2005.

(51) Int. Cl. A61K 31/495; C07D 471/04

(52) U.S. Cl. 514/249; 544/350

(57) ABSTRACT

Substituted hydroxytetrahydropyrrolopyrazinone and substituted hydroxytetrahydro-pyrazolopyrazinone compounds of Formula (I) are inhibitors of HIV integrase and inhibitors of HIV replication: (I) wherein R¹, R², R³, R⁴; R⁵, X, ring A, and Q are as defined herein. The compounds are useful in the prevention and treatment of infection by HIV and in the prevention, delay in the onset, and treatment of AIDS. The compounds are employed against HIV infection and AIDS as compounds per se or in the form of pharmaceutically acceptable salts. The compounds and their salts can be employed as ingredients in pharmaceutical compositions, optionally in combination with other antivirals, immunomodulators, antibiotics or vaccines.

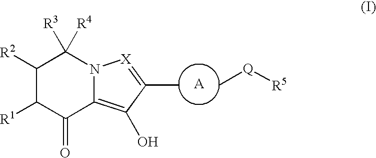

The present invention is directed to substituted hydroxytetrahydropyrrolopyrazinone and substituted hydroxytetrahydropyrazolopyrazinone compounds and pharmaceutically acceptable salts thereof, their synthesis, and their use as inhibitors of the HIV integrase enzyme. The compounds and pharmaceutically acceptable salts thereof of the present invention are useful for preventing or treating infection by HIV and for preventing or treating or delaying the onset of AIDS.

### BACKGROUND OF THE INVENTION

A retrovirus designated human immunodeficiency virus (HIV), particularly the strains known as HIV type-1 (HIV-1) virus and type-2 (HIV-2) virus, is the etiological agent of the complex disease that includes progressive destruction of the immune system (acquired immune deficiency syndrome; AIDS) and degeneration of the central and peripheral nervous system. This virus was previously known as LAV, HTLV-III, or ARV. A common feature of retrovirus replication is the insertion by virally-encoded integrase of +proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277 (1985)]. Amino acid sequence homology provides evidence that the pol sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhibitors such as indinavir and nelfinavir. The compounds of this invention are inhibitors of HIV integrase and inhibitors of HIV replication. The inhibition of integrase in vitro and HIV replication in cells is a direct result of inhibiting the strand transfer reaction catalyzed by the recombinant integrase in vitro in HIV infected cells. The particular advantage of the present invention is highly specific inhibition of HIV integrase and HIV replication.

### SUMMARY OF THE INVENTION

The present invention is directed to substituted hydroxytetrahydropyrrolopyrazinones and substituted hydroxytetrahydropyrazolopyrazinones. These compounds are useful in the inhibition of HIV integrase, the prevention of infection by HIV, the treatment of infection by HIV and in the prevention, treatment, and delay in the onset of AIDS and/or ARC, either as compounds or their pharmaceutically acceptable salts or hydrates (when appropriate), or as pharmaceutical composition ingredients, whether or not in combination with other HIV/AIDS antivirals, anti-infectives, immunomodulators, antibiotics or vaccines. More particularly, the present invention includes compounds of Formula (I) and pharmaceutically acceptable salts and hydrates thereof:

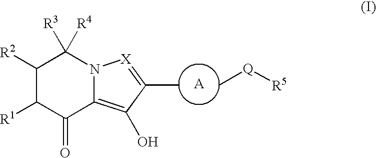

wherein
R¹ is —H, —C₁₋₆ alkyl, —C₃₋₆ cycloalkyl, or —C₁₋₆ alkyl which is substituted with 1 or 2 substituents each of which is independently:
(1) C₃₋₈ cycloalkyl,
(2) aryl,
(3) a 5- or 6-membered saturated or mono-unsaturated heterocyclic ring containing from 1 to 4 heteroatoms independently selected from N, O and S,
(4) a 5- or 6-membered heteroaromatic ring containing from 1 to 4 heteroatoms independently selected from N, O and S, or
(5) a 9- or 10-membered fused bicyclic heterocycle containing from 1 to 4 heteroatoms independently selected from N, O and S, wherein at least one of the rings is aromatic;
wherein
(A) each cycloalkyl is optionally substituted with from 1 to 3 substituents, each of which is independently halo, —C₁₋₆ alkyl, —O—C₁₋₆ alkyl, —OH, or C₁₋₆ haloalkyl;
(B) each aryl is optionally substituted with from 1 to 5 substituents each of which is independently
   (1) —C₁₋₆ alkyl, optionally substituted with from 1 to 3 substituents each of which is independently —OH, —O—C₁₋₆ alkyl, —O—C₁₋₆ haloalkyl, —CN, —NO₂, —N(R^{a}R^{b}), —C(═O)N(R^{a}R^{b}), —C(═O)R^{a}, —CO₂R^{c}, —SH, —S(O)ₙR^{d}, —SO₂N(R^{a}R^{b}), —N(R^{a})C(═O)R^{b}, —N(R^{a})CO₂R^{d}, —N(R^{a})SO₂R^{d}, —N(R^{a})SO₂N(R^{a}R^{b}), —OC(═O)N(R^{a}R^{b}), or N(R^{a})C(═O)N(R^{a}R^{b}),
   (2) —O—C₁₋₆ alkyl, optionally substituted with from 1 to 3 substituents each of which is independently —OH, —O—C₁₋₆ alkyl, —O—C₁₋₆ haloalkyl, —SH, —S(O)ₙR^{d}, —C(═O)N(R^{a}R^{b}), —SO₂N(R^{a}R^{b}), —N(R^{a})C(═O)R^{b}, —N(R^{a})CO₂R^{d}, —N(R^{a})SO₂R^{d}, —N(R^{a})SO₂N(R^{a}R^{b}), —OC(═O)N(R^{a}R^{b}), or —N(R^{a})C(═O)N(R^{a}R^{b}),
   (3) —C₁₋₆ haloalkyl,
   (4) —O—C₁₋₆ haloalkyl,
   (5) —OH,
   (6) halo,
   (7) —CN,
   (8) —NO₂,
   (9) —N(R^{a}R^{b}),
   (10) —C(═O)N(R^{a}R^{b}),
   (11) —C(═O)R^{a},
   (12) —CO₂R^{c},
   (13) —SR^{c},
   (14) —S(═O)R^{d},
   (15) —SO₂R^{d},
   (16) —N(R^{a})SO₂R^{d},
   (17) —SO₂N(R^{a}R^{b}),
   (18) —N(R^{a})C(═O)R^{b}, or
   (19) —N(R^{a})CO₂R^{d};
(C) each saturated or mono-unsaturated heterocyclic ring is
   (i) optionally substituted with from 1 to 5 substituents each of which is independently halogen, —C₁₋₆ alkyl, —C₁₋₆ haloalkyl, —O—C₁₋₆ alkyl, —O—C₁₋₆ haloalkyl, or oxo; and
   (ii) optionally substituted with 1 or 2 substituents each of which is independently aryl or a 5- or 6-membered heteroaromatic ring containing from 1 to 4 heteroatoms independently selected from N, O and S; and
(D) each heteroaromatic ring or each fused bicyclic heterocycle is
   (i) optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, or oxo; and
   (ii) optionally substituted with 1 or 2 substituents each of which is independently aryl or —C₁₋₆ alkyl-aryl;
      R² is —H or —C₁₋₆ alkyl;
      R³ is —H, —C₁₋₆ alkyl, —C₁₋₆ haloalkyl, or —C₁₋₆ alkyl substituted with one of —OH, —O—C₁₋₆ alkyl, —O—C₁₋₆ haloalkyl, —CN, —NO₂, —N(R^{a}R^{b}), —C(═O)N(R^{a}R^{b}), —C(═O)R^{a}, —CO₂R^{c}, —SH, —S(O)ₙR^{d}, —SO₂N(R^{a}R^{b}), —N(R^{a})C(═O)R^{b}, —N(R^{a})CO₂R^{d}, —N(R^{a})SO₂R^{d}, —N(R^{a})SO₂N(R^{a}R^{b}), —OC(═O)N(R^{a}R^{b}), or —N(R^{a})C(═O)N(R^{a}R^{b});

### R⁴ is:

(1) —H,
(2) —C₁₋₆ alkyl optionally substituted with one of —OH, —O—C₁₋₆ alkyl, —O—C₁₋₆ haloalkyl, —CN, —NO₂, —N(R^{a}R^{b}), —C(═O)N(R^{a}R^{b}), —C(═O)R^{a}, —CO₂R^{c}, —SH, —S(O)ₙR^{d}, —SO₂N(R^{a}R^{b}), —N(R^{a})—C(R^{b})═O, —N(R^{a})SO₂R^{d}, —N(R^{a})SO₂N(R^{a}R^{b}), —OC(═O)N(R^{a}R^{b}), —N(R^{a})C(═O)N(R^{a}R^{b}), —O—C₁₋₆ alkyl-C(═O)N(R^{a}R^{b}), —S—C₁₋₆ alkyl-C(═O)N(R^{a}R^{b}), —N(R^{a})—C₁₋₆ alkyl-C(═O)N(R^{a}R^{b}), or —N(SO₂R^{d})—C₁₋₆ alkyl-C(═O)N(R^{a}R^{b}),
(3) —C₁₋₆ haloalkyl,
(4) —C(═O)R^{a},
(5) —CO₂R^{c},
(6) —C(═O)N(R^{a}R^{b}),
(7) —SO₂N(R^{a}R^{b}),
(8) —C₂₋₆ alkenyl,
(9) —C₂₋₆ alkenyl-C(═O)—N(R^{a})₂,
(10) —C₂₋₅ alkynyl,
(11) —C₂₋₅ alkynyl-CH₂N(R^{a})₂,
(12) —C₂₋₅ alkynyl-CH₂OR^{a},
(13) —C₂₋₅ alkynyl-CH₂S(O)ₙR^{c}, or
(14) —R^{j},
(15) —C₁₋₆ alkyl substituted with R^{j},
(16) —C₁₋₆ haloalkyl substituted with R^{j},
(17) —C₁₋₆ alkyl-O—R^{j},
(18) —C₁₋₆ alkyl-O—C₁₋₆ alkyl-R^{j},
(19) —C₁₋₆ alkyl-S(O)ₙ—R^{j},
(20) —C₁₋₆ alkyl-S(O)ₙ—C₁₋₆ alkyl-R^{j},
(21) —C₁₋₆ alkyl-N(R^{a})—R^{j},
(22) —C₁₋₆ alkyl-N(R^{a})—C₁₋₆ alkyl-R^{j},
(23) —C₁₋₆ alkyl-N(R^{a})—C₁₋₆ alkyl-OR^{j}, with the proviso that the —N(R^{a})— moiety and the —OR^{j} moiety are not both attached to the same carbon of the —C₁₋₆ alkyl-moiety,
(24) —C₁₋₆ alkyl-C(═O)—R^{j},
(25) —C₁₋₆ alkyl-C(═O)N(R^{a})—R^{j},
(26) —C₁₋₆ alkyl-N(R^{a})C(═O)—R^{j},
(27) —C₁₋₆ alkyl-C(═O)N(R^{a})—C₁₋₆ alkyl-R^{j}, or
(28) —C₁₋₆ alkyl-N(R^{a})—C₀₋₆ alkyl-S(O)ₙRⁱ;
   wherein R^{j} is
   (i) aryl, which is optionally substituted with from 1 to 5 substituents each of which is independently —C₁₋₆ alkyl, —C₁₋₆ alkyl-OH, —C₁₋₆ alkyl-O—C₁₋₆ alkyl, —C₁₋₆ alkyl-O—C₁₋₆ haloalkyl, —C₁₋₆ alkyl-N(R^{a}R^{b}), —C₁₋₆ alkyl-C(═O)N(R^{a}R^{b}), —C₁₋₆ alkyl-C(═O)R^{a}, —C₁₋₆ alkyl-CO₂R^{c}, —C₁₋₆ alkyl-S(O)ₙR^{c}, —O—C₁₋₆ alkyl, —C₁₋₆ haloalkyl, —O—C₁₋₆ haloalkyl, —OH, halo, —N(R^{a}R^{b}), —C(═O)N(R^{a}R^{b}), —C(═O)R^{a}, —CO₂R^{c}, —SH, —S(O)ₙR^{d}, or —SO₂N(R^{a}R^{b});
   (ii) a 4- to 7-membered saturated or mono-unsaturated heterocyclic ring containing at least one carbon atom and from 1 to 4 heteroatoms independently selected from N, O and S, wherein the heterocyclic ring is:
      (a) optionally substituted with from 1 to 5 substituents each of which is independently halogen, —C₁₋₆ alkyl, —C₁₋₆ haloalkyl, —O—C₁₋₆ alkyl, —O—C₁₋₆ haloalkyl, or oxo; and
      (b) optionally mono-substituted with aryl or HetA;
         wherein HetA is a 5- or 6-membered heteroaromatic ring containing from 1 to 4 heteroatoms independently selected from N, O and S, wherein the heteroaromatic ring is optionally fused with a benzene ring, and HetA is optionally substituted with from 1 to 4 substituents each of which is independently —C₁₋₆ alkyl, —C₁₋₆ haloalkyl, —O—C₁₋₆ alkyl, —O—C₁₋₆ haloalkyl, or oxo; or
   (iii) a 5- or 6-membered heteroaromatic ring containing from 1 to 4 heteroatoms independently selected from N, O and S, wherein the heteroaromatic ring is optionally substituted with from optionally substituted with from 1 to 4 substituents each of which is independently —C₁₋₆ alkyl, —C₁₋₆ haloalkyl, —O—C₁₋₆ alkyl, —O—C₁₋₆ haloalkyl, or oxo;
      ring A is a 5- or 6-membered saturated, partially saturated, or aromatic monocyclic ring or a 8- to 11-membered saturated, partially saturated, or aromatic bicyclic ring, wherein said monocyclic or bicyclic ring contains from 1 to 4 heteroatoms independently selected from N, O and S;
      Q is C₁₋₆ alkylene, —NR⁶—, —O—, —C(O)—, —CH(OR⁶)—, —S(O)₂—, or —CF₂—;

### R⁵ is

(1) C₃₋₈ cycloalkyl wherein said cycloalkyl is optionally substituted with aryl and said cycloalkyl is optionally substituted with from 1 to 3 substituents, each of which is independently halo, —C₁₋₆ alkyl, —O—C₁₋₆ alkyl, —OH, or C₁₋₆ haloalkyl,
(2) aryl,
(3) a fused bicyclic carbocycle consisting of a benzene ring fused to a C₅₋₇ cycloalkyl,
(4) a 5- or 6-membered saturated or partially saturated heterocyclic ring containing from 1 to 4 heteroatoms independently selected from N, O and S,
(5) a 5- or 6-membered heteroaromatic ring containing from 1 to 4 heteroatoms independently selected from N, O and S, or
(6) a 9- or 10-membered fused bicyclic heterocyclic ring containing from 1 to 4 heteroatoms independently selected from N, O and S, wherein at least one of the rings is aromatic;
wherein
   each aryl in (1) or (2) or each fused carbocycle in (3) is optionally substituted with one or more substituents (e.g., optionally from 1 to 5, or 1 to 4, or 1 to 3, or 1 or 2 substituents; or is optionally mono-substituted) each of which is independently halogen, —OH, —C₁₋₆ alkyl, —C₁₋₆ alkyl-OR^{a}, —C₁₋₆ haloalkyl, —O—C₁₋₁₆ alkyl, —O—C₁₋₆ haloalkyl, —CN, —NO₂, —N(R^{a}R^{b}), —C₁₋₆ alkyl-N(R^{a}R^{b}), —C(═O)N(R^{a}R^{b}), —C(═O)R^{a}, —CO₂R^{a}, —C₁₋₆ alkyl-CO₂R^{a}, —OCO₂R^{a}, —SR^{a}, —S(═O)R^{a}, —SO₂R^{a}, —N(R^{a})SO₂R^{b}, —SO₂N(R^{a}R^{b}), —N(R^{a})C(═O)R^{b}, —N(R^{a})CO₂R^{b}, —C₁₋₆ alkyl-N(R^{a})CO₂R^{b}, aryl, —C₁₋₆ alkyl-aryl, —O-aryl, or —C₀₋₆ alkyl-het wherein het is a 5- or 6-membered heteroaromatic ring containing from 1 to 4 heteroatoms independently selected from N, O and S, and het is optionally fused with a benzene ring, and is optionally substituted with one or more substituents (e.g., optionally from 1 to 5, or 1 to 4, or 1 to 3, or 1 or 2 substituents; or is optionally mono-substituted) each of which is independently —C₁₋₆ alkyl, —C₁₋₆ haloalkyl, —O—C₁₋₆ alkyl, —O—C₁₋₆ haloalkyl, oxo, or —CO₂R^{a};
   each saturated or unsaturated non-aromatic heterocyclic ring in (4) is optionally substituted with one or more substituents (e.g., optionally from 1 to 6, or 1 to 5, or 1 to 4, or 1 to 3, or 1 or 2 substituents; or is optionally mono-substituted) each of which is independently halogen, —C₁₋₆ alkyl, —C₁₋₆ haloalkyl, —O—C₁₋₆ alkyl, —O—C₁₋₆ haloalkyl, oxo, aryl, or a 5- or 6-membered heteroaromatic ring containing from 1 to 4 heteroatoms independently selected from N, O and S; and
   each heteroaromatic ring in (5) or each fused bicyclic heterocycle in (6) is optionally substituted with one or more substituents (e.g., optionally from 1 to 6, or 1 to 5, or 1 to 4, or 1 to 3, or 1 or 2 substituents; or is optionally mono-substituted) each of which is independently halogen, —C₁₋₆ alkyl, —C₁₋₆ haloalkyl, —O—C₁₋₆ alkyl, —O—C₁₋₆ haloalkyl, oxo, aryl, or —C₁₋₆ alkyl-aryl;
      R⁶ is —H, —C₁₋₆ alkyl, C₃₋₈ cycloalkyl, —C₁₋₆ haloalkyl, aryl, ar(C₁₋₃)alkyl, or HetB;
      HetB is a 3- or 7-membered saturated, partially saturated, or aromatic monocyclic ring or a 8- to 11-membered saturated, partially saturated, or aromatic bicyclic ring, wherein said monocyclic or bicyclic ring contains from 1 to 4 heteroatoms independently selected from N, O and S;
      each R^{a}, R^{b}, and R^{c} is independently —H or —C₁₋₆ alkyl;
      each n is independently an integer equal to zero, 1 or 2;

### X is N or C(R⁷);

R⁷ is —H or —C₁₋₆ alkyl; and
each R^{d} is independently —C₁₋₆ alkyl.

The present invention also includes pharmaceutical compositions containing a compound of the present invention and methods of preparing such pharmaceutical compositions. The present invention further includes methods of treating AIDS, methods of delaying the onset of AIDS, methods of preventing AIDS, methods of preventing infection by HIV, and methods of treating infection by HIV.

Other embodiments, aspects and features of the present invention are either further described in or will be apparent from the ensuing description, examples and appended claims.

### DETAILED DESCRIPTION OF THE INVENTION

The present invention includes compounds of Formula (I) above, and pharmaceutically acceptable salts and hydrates thereof. These compounds and their pharmaceutically acceptable salts and hydrates are HIV integrase inhibitors (e.g., HIV-1 integrase inhibitors). The present invention also includes compounds of Formula (I-a) and Formula (I-b) wherein all variables are as defined for Formula (I).

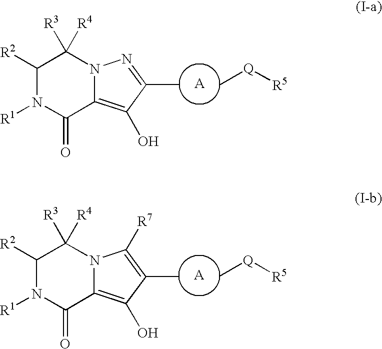

An embodiment of the present invention is a compound of Formula (I), (I-a) or (1-b), or a pharmaceutically acceptable salt thereof, wherein R¹ is H or —C₁₋₆ alkyl; and all other variables are as originally defined (i.e., as defined in the Summary of the Invention). In an aspect of this embodiment, R¹ is —C₁₋₃ alkyl. In another aspect of this embodiment, R¹ is methyl.

Another embodiment of the present invention is a compound of Formula (I), (I-a) or (1-b), or a pharmaceutically acceptable salt thereof, wherein R¹ is —C₁₋₄ allyl mono-substituted with aryl; wherein the aryl is optionally substituted with from 1 to 4 substituents each of which is independently
(1) —C₁₋₄ alkyl, optionally mono-substituted with —OH, —O—C₁₋₄ alkyl, —O—C₁₋₄ haloalkyl, —CN, —N(R^{a}R^{b}), —C(═O)N(R^{a}R^{b}), —C(═O)R^{a}, —CO₂R^{c}, —SH, —S(O)ₙR^{d}, —SO₂N(R^{a}R^{b}), —N(R^{a})C(═O)R^{b}, —N(R^{a})CO₂R^{d}, —N(R^{a})SO₂R^{d}, —N(R^{a})SO₂N(R^{a}R^{b}), —OC(═O)N(R^{a}R^{b}), or —N(R^{a})C(═O)N(R^{a}R^{b}),
(2) —O—C₁₋₄ alkyl, optionally mono-substituted with —OH, —O—C₁₋₄ alkyl, —O—C₁₋₄ haloallyl, —SH, —S(O)ₙR^{d}, —N(R^{a})—CO₂R^{d}, —C(═O)N(R^{a}R^{b}), —SO₂N(R^{a}R^{b}), —N(R^{a})C(═O)R^{b}, —N(R^{a})CO₂R^{d}, —N(R^{a})SO₂R^{d}, —N(R^{a})SO₂N(R^{a}R^{b}), —OC(═O)N(R^{a}R^{b}), or —N(R^{a})C(═O)N(R^{a}R^{b}),
(3) —C₁₋₄ haloalkyl,
(4) —O—C₁₋₄ haloalkyl,
(5) —OH,
(6) halo,
(7) —CN,
(8) —NO₂,
(9) —N(R^{a}R^{b}),
(10) —SR^{c},
(11) —S(═O)R^{d},
(12) —SO₂R^{d},
(13) —N(R^{a})SO₂R^{d},
(14) —SO₂N(R^{a}R^{b}),
(15) —N(R^{a})C(═O)R^{b}, or
(16) —N(R^{a})CO₂R^{d};
   and all other variables are as originally defined above.

Another embodiment of the present invention is a compound of Formula (I), (I-a) or (1-b), or a pharmaceutically acceptable salt thereof, wherein R¹ is —(CH₂)₁₋₄-phenyl, wherein the phenyl is optionally substituted with from 1 to 3 substituents each of which is independently
(1) —C₁₋₄ alkyl, optionally mono-substituted with —OH, —O—C₁₋₄ alkyl, —O—C₁₋₄ haloalkyl, —CN, —N(R^{a}R^{b}), —C(═O)N(R^{a}R^{b}), —C(═O)R^{a}, —CO₂R^{c}, —SH, —S(O)ₙR^{d}, or —SO₂N(R^{a}R^{b}),
(2) —O—C₁₋₄ alkyl,
(3) —C₁₋₁₄ haloalkyl,
(4) —O—C₁₋₄ haloalkyl,
(5) —OH,
(6) halo,
(7) —CN,
(8) —NO₂,
(9) N(R^{a}R^{b}),
(10) —SR^{C},
(11) —S(═O)R^{d},
(12) —SO₂R^{d},
(13) —N(R^{a})SO₂R^{d},
(14) —SO₂N(R^{a}R^{b}),
(15) —N(R^{a})C(═O)R^{b}, or
(16) —N(R^{a})CO₂R^{d};
   and all other variables are as originally defined above.

In an aspect of the preceding embodiment, R¹ is

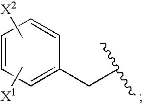

wherein X¹ and X² are each independently
(1) —H,
(2) methyl,
(3) ethyl,
(4) methoxy,
(5) ethoxy,
(6) —CF₃,
(7) fluoro,
(8) bromo, or
(9) chloro.
   In a feature of the preceding aspect, R¹ is 4-fluorobenzyl.

Another embodiment of the present invention is a compound of Formula (I), (I-a) or (1-b), or a pharmaceutically acceptable salt thereof, wherein R² is —H or —C₁₋₄ alkyl; and all other variables are as originally defined or as defined in any of the preceding embodiments. In an aspect of this embodiment, R² is —H.

Another embodiment of the present invention is a compound of Formula (I), (I-a) or (1-b), or a pharmaceutically acceptable salt thereof, wherein R³ is —H or —C₁₋₄ alkyl; and all other variables are as originally defined or as defined in any of the preceding embodiments. In an aspect of this embodiment, R³ is —H.

Another embodiment of the present invention is a compound of Formula (I), (I-a) or (1-b), or a pharmaceutically acceptable salt thereof, wherein R⁴ is:
(1) —H,
(2) —C₁₋₄ alkyl optionally substituted with one of —OH, —O—C₁₋₄ alkyl, —O—C₁₋₁₄ haloalkyl, —CN, —N(R^{a}R^{b}), —C(═O)N(R^{a}R^{b}), —C(═O)R^{a}, —CO₂R^{c}, —SH, —S(O)ₙR^{d}, —SO₂N(R^{a}R^{b}), —N(R^{a})—C(R^{b})═O, —N(R^{a})SO₂R^{b}, or —N(R^{a})SO₂N(R^{a}R^{b}),
(3) —C(═O)N(R^{a}R^{b}),
(4) —R^{j},
(5) —C₁₋₄ alkyl substituted with R^{j},
(6) —C₁₋₄ alkyl-O—R^{j}, or
(7) —C₁₋₄ alkyl-O—C₁₋₄ alkyl-R^{j};
   and all other variables are as originally defined or as defined in any of the preceding embodiments.

In an aspect of the immediately preceding embodiment, R⁴ is:
(1) —H,
(2) —C₁₋₄ alkyl optionally substituted with one of —OH, —N(R^{a}R^{b}), or —C(═O)N(R^{a}R^{b}),
(3) —C(═O)N(R^{a}R^{b}),
(4) —(CH₂)₁₋₃—R^{j},
(5) —(CH₂)₁₋₃—O—R^{j},
(6) —(CH₂)₁₋₃—O—(CH₂)₁₋₃—R^{j}, or
(7) —R^{j}.

In a feature of the preceding aspect, R^{j} is:
(i) phenyl, which is optionally substituted with from 1 to 3 substituents each of which is independently —C₁₋₄ alkyl, —C₁₋₄ alkyl-OH, —C₁₋₄ alkyl-O—C₁₋₄ alkyl, —C₁₋₄ alkyl-O—C₁₋₄ haloalkyl, —C₁₋₄ alkyl-N(R^{a}R^{b}), —C₁₋₄ alkyl-C(═O)N(R^{a}R^{b}), —C₁₋₄ alkyl-C(═O)R^{a}, —C₁₋₄ alkyl-CO₂R^{c}, —C₁₋₄ alkyl-S(O)ₙR^{d}, —O—C₁₋₄ alkyl, —C₁₋₄ haloalkyl, —O—C₁₋₄ haloalkyl, —OH, halo, —N(R^{a}R^{b}), —C(═O)N(R^{a}R^{b}), —C(═O)R^{a}, —CO₂R^{c}, —SH, —S(O)ₙR^{d}, or —SO₂N(R^{a}R^{b});
(ii) a 4- to 7-membered saturated heterocyclic ring containing at least one carbon atom and a total of from 1 to 4 heteroatoms independently selected from 1 to 4 N atoms, from 0 to 20 atoms, and from 0 to 2 S atoms, wherein the heterocyclic ring is:
   (a) optionally substituted with from 1 to 4 substituents each of which is independently halogen, —C₁₋₄ alkyl, —C₁₋₄ haloalkyl, —O—C₁₋₄ alkyl, —O—C₁₋₄ haloalkyl, or oxo; and
   (b) optionally mono-substituted with phenyl or HetA;
      wherein HetA is a 5- or 6-membered heteroaromatic ring containing a total of from 1 to 4 heteroatoms independently selected from 1 to 4 N atoms, from 0 to 20 atoms, and from 0 to 2 S atoms, wherein HetA is optionally substituted with from 1 to 3 substituents each of which is independently —C₁₋₄ alkyl, —C₁₋₄ haloalkyl, —O—C₁₋₄ alkyl, —O—C₁₋₁₄ haloalkyl, or oxo; or
(iii) a 5- or 6-membered heteroaromatic ring containing a total of from 1 to 4 heteroatoms independently selected from 1 to 4 N atoms, from 0 to 20 atoms, and from 0 to 2 S atoms, wherein the heteroaromatic ring is optionally substituted with from optionally substituted with from 1 to 3 substituents each of which is independently —C₁₋₆ alkyl, —C₁₋₆ haloalkyl, —O—C₁₋₆ alkyl, —O—C₁₋₆ haloalkyl, or oxo.
   In another feature of the preceding aspect, HetA is a 5- or 6-membered heteroaromatic ring containing 1 or 2 N atoms, wherein HetA is optionally substituted with from 1 to 3 substituents each of which is independently —C₁₋₄ alkyl, —C₁₋₄ haloalkyl, —O—C₁₋₄ alkyl, —O—C₁₋₄ haloalkyl, or oxo. In yet another feature of the preceding aspect, HetA is pyrrolyl, pyrazolyl, imidazolyl, pyridyl, or pyrazinyl; which is optionally substituted with from 1 to 3 substituents each of which is independently —C₁₋₄ alkyl (e.g., methyl), —C₁₋₄ haloalkyl (e.g., trifluoromethyl), —O—C₁₋₄ alkyl (e.g., methoxy), —O—C₁₋₄ haloalkyl (e.g., —OCF₃), or oxo.

Another embodiment of the present invention is a compound of Formula (I), (I-a) or (1-b), or a pharmaceutically acceptable salt thereof, wherein R^{j} is:
(i) phenyl, which is optionally substituted with from 1 to 3 substituents each of which is independently —C₁₋₄ alkyl, —C₁₋₄ alkyl-OH, —C₁₋₄ alkyl-O—C₁₋₄ alkyl, —C₁₋₄ alkyl-O—C₁₋₄ haloalkyl, —C₁₋₄ alkyl-N(R^{a}R^{b}), —C₁₋₄ alkyl-C(═O)N(R^{a}R^{b}), —C₁₋₄ alkyl-C(═O)R^{a}, —C₁₋₄ alkyl-CO₂R^{c}, —C₁₋₄ alkyl-S(O)ₙR^{d}, —O—C₁₋₄ alkyl, —C₁₋₄ haloalkyl, —O—C₁₋₄ haloalkyl, —OH, halo, —N(R^{a}R^{b}), —C(═O)N(R^{a}R^{b}), —C(═O)R^{a}, —CO₂R^{c}, —SH, —S(O)ₙR^{d}, or —SO₂N(R^{a}R^{b}); or
(ii) a saturated heterocyclic ring selected from the group consisting of piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, thiadiazepanyl, dithiazepanyl, azepanyl, diazepanyl, thiadiazinanyl, and dioxanyl; wherein the saturated heterocyclic ring is:
   (a) optionally substituted with from 1 to 4 substituents each of which is independently halogen, —C₁₋₄ alkyl, —C₁₋₄ haloalkyl, —O—C₁₋₄ alkyl, —O—C₁₋₄ haloalkyl, or oxo; and
   (b) optionally mono-substituted with phenyl or HetA; wherein HetA is a heteroaromatic ring selected from the group consisting of pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl; wherein the heteroaromatic ring is optionally substituted with from 1 to 3 substituents each of which is independently —C₁₋₄ alkyl, —C₁₋₄ haloalkyl, —O—C₁₋₄ alkyl, —O—C₁₋₄ haloalkyl, or oxo;
      and all other variables are as originally defined or as defined in any of the preceding embodiments.

Another embodiment of the present invention is a compound of Formula (I), (I-a) or (I-b), or a pharmaceutically acceptable salt thereof, wherein ring A is a 5- or 6-membered saturated, partially saturated, or aromatic monocyclic ring containing from 1 to 4 heteroatoms independently selected from N, O and S; and all other variables are as originally defined or as defined in any one of the preceding embodiments.

In an aspect of the preceding embodiment, ring A is a 5- or 6-membered aromatic monocyclic ring containing from 1 to 4 heteroatoms independently selected from N, O and S. In a feature of the preceding aspect, ring A is oxadiazolyl, triazolyl, thiadazolyl, oxazolyl, tetrazolyl, or pyrimidinyl. In another feature of the preceding aspect, ring A is 1,2,4-oxadiazolyl, 1,3,4-oxadiazolyl, 1,2,4-triazolyl, 1,3,4-thiadazolyl, 1,3-oxazol-2-yl, 2-tetrazol-5-yl, 1-tetrazol-5-yl or 4-pyrimidinyl.

Another embodiment of the present invention is a compound of Formula (I), (I-a) or (1-b), or a pharmaceutically acceptable salt thereof, wherein Q is C₁₋₆ alkylene, —CH(OR⁶)— or —CF₂—; and all other variables are as originally defined or as defined in any one of the preceding embodiments. In an aspect of this embodiment, Q is C₁₋₃ alkylene, —CH(OR⁶)— wherein R⁶ is H or —CF₂—. In another aspect of this embodiment, Q is C₁₋₃ alkylene. In a feature of the preceding aspect, Q is —CH₂—.

Another embodiment of the present invention is a compound of Formula (I), (I-a) or (1-b), or a pharmaceutically acceptable salt thereof, wherein X is N or CH (i.e., R⁷ is —H); and all other variables are as originally defined or as defined in any of the preceding embodiments.

Another embodiment of the present invention is a compound of Formula (I), (I-a) or (I-b), or a pharmaceutically acceptable salt thereof, wherein R⁵ is aryl which is optionally substituted with from 1 to 4 substituents each of which is independently halogen, —OH, —C₁₋₄ alkyl, —C₁₋₄ alkyl-OR^{a}, —C₁₋₄ haloalkyl, —O—C₁₋₄ alkyl, —O—C₁₋₄ haloalkyl, —CN, —NO₂, —N(R^{a}R^{b}), —C₁₋₄ alkyl-N(R^{a}R^{b}), —C(═O)N(R^{a}R^{b}), —C(═O)R^{a}, —CO₂R^{a}, —C₁₋₄ alkyl-CO₂R^{a}, —OCO₂R^{a}, —SR^{a}, —S(═O)R^{a}, —SO₂R^{a}, —N(R^{a})SO₂R^{b}, —SO₂N(R^{a}R^{b}), —N(R^{a})C(═O)R^{b}, —N(R^{a})CO₂R^{b}, —C₁₋₄ alkyl-N(R^{a})CO₂R^{b}, methylenedioxy attached to two adjacent ring carbon atoms, phenyl, —C₁₋₄ alkyl-phenyl, —O-phenyl, or (CH₂)₀₋₂-het;

wherein het is a 5- or 6-membered heteroaromatic ring containing from 1 to 4 heteroatoms independently selected from N, O and S, and het is optionally fused with a benzene ring, and is optionally substituted with 1 or 2 substituents each of which is independently —C₁₋₄ allyl, —C₁₋₄ haloalkyl, —O—C₁₋₄ alkyl, —O—C₁₋₄ haloalkyl, or —CO₂R^{a}; and all other variables are as originally defined or as defined in any one of the preceding embodiments.

An aspect of the preceding embodiment is a compound of Formula (I), (I-a) or (1-b), or a pharmaceutically acceptable salt thereof, wherein R⁵ is phenyl, wherein the phenyl is optionally substituted with from 1 to 3 substituents each of which is independently fluoro, bromo, chloro, —OH, —C₁₋₄ alkyl, —C₁₋₄ fluoroalkyl, —O—C₁₋₄ alkyl, —O—C₁₋₄ fluoroalkyl, —(CH₂)₁₋₂—N(R^{a}R^{b}), —SO₂R^{a}, —(CH₂)₀₋₂—CO₂R^{a}, —(CH₂)₀₋₂—N(R^{a})CO₂R^{b}, —NO₂, —SR^{a}, —N(R^{a}R^{b}) or phenyl; and each R^{a} and R^{b} is independently is H or —C₁₋₄ alkyl; and all other variables are as originally defined or as defined in any one of the preceding embodiments.

In a feature of the preceding aspect, R⁵ is phenyl, wherein the phenyl is optionally substituted with from 1 to 3 substituents, each of which is independently —F, —Br, —Cl, —OH, —C₁₋₄ alkyl, —C₁₋₄ fluoroalkyl, —O—C₁₋₄ alkyl, —SO₂—C₁₋₄ alkyl, —S—C₁₋₄ alkyl, —N(CH₃)₂ or —O—C₁₋₄ fluoroalkyl. In another feature of the preceding aspect, R⁵ is p-fluorophenyl or 2,3-dimethoxyphenyl.

In another feature of the preceding aspect, R⁵ is phenyl, wherein the phenyl is optionally substituted with from 1 to 3 substituents each of which is independently fluoro, bromo, chloro, —OH, —C₁₋₄ alkyl, —C₁₋₄ fluoroalkyl, —O—C₁₋₄ alkyl, —O—C₁₋₄ fluoroalkyl, —(CH₂)₁₋₂—N(R^{a}R^{b}), —SO₂R^{a}, —(CH₂)₀₋₂—CO₂R^{a}, —(CH₂)₀₋₂—N(R^{a})CO₂R^{b}, —NO₂, or phenyl. In yet another feature of the preceding aspect, the phenyl is optionally substituted with from 1 to 3 substituents, each of which is independently —F, —Br, —Cl, —OH, —C₁₋₄ alkyl, —C₁₋₄ fluoroalkyl, —O—C₁₋₄ alkyl, or —O—C₁₋₄ fluoroalkyl.

A class of the present invention includes a compound of Formula (I-1), or a pharmaceutically acceptable salt thereof, wherein:

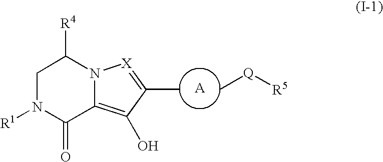

R¹ is —C₁₋₆ alkyl or

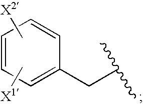

### R⁴ is:

(1) —H,
(2) —C₁₋₄ alkyl optionally substituted with one of —OH, —N(R^{a}R^{b}), or —C(═O)N(R^{a}R^{b}),
(3) —C(═O)N(R^{a}R^{b}),
(4) —R^{j},
(5) —(CH₂)₁₋₃—RJ,
(6) —(CH₂)₁₋₃—O—R^{j}, or
(7) —(CH₂)₁₋₃—O—(CH₂)₁₋₃—R^{j};

### X is N or CH;

ring A is a 5- or 6-membered aromatic monocyclic ring containing from 1 to 4 heteroatoms independently selected from N, O and S;
Q is —C₁₋₃ alkylene;

### R⁵ is:

(1) phenyl, wherein the phenyl is optionally substituted with from 1 to 3 substituents each of which is independently fluoro, bromo, chloro, —OH, —C₁₋₄ alkyl, —C₁₋₄ fluoroalkyl, —O—C₁₋₄ alkyl, —O—C₁₋₄ fluoroalkyl, —(CH₂)₁₋₂—N(R^{a}R^{b}), —SO₂R^{a}, —(CH₂)₀₋₂—CO₂R^{a}, —(CH₂)₀₋₂—N(R^{a})CO₂R^{b}, —NO₂, —SR^{a}, —N(R^{a}R^{b}) or phenyl; or
(2) a fused bicyclic carbocycle selected from

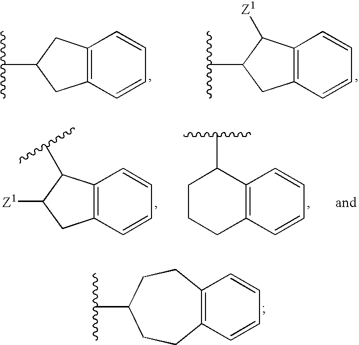

wherein Z¹ is —H or —OH; and
   X¹′ and X²′ are each independently:
(1) —H,
(2) C₁₋₄ alkyl,
(2) —O—C₁₋₄ alkyl,
(3) —C₁₋₄ haloalkyl,
(4) —O—C₁₋₄ haloalkyl, or
(5) halo;
   and all other variables are as originally defined above.

A sub-class of the preceding class of compounds of the present invention includes a compound of Formula (I-1), or a pharmaceutically acceptable salt thereof, wherein:

R¹ is —C₁₋₃ alkyl;

### R⁴ is H or —R^{j}; and

R⁵ is phenyl, wherein the phenyl is optionally substituted with from 1 to 3 substituents each of which is independently fluoro, bromo, chloro, —OH, —C₁₋₄ alkyl, —C₁₋₄ fluoroalkyl, —O—C₁₋₄ alkyl, —O—C₁₋₄ fluoroalkyl, —(CH₂)₁₋₂—N(R^{a}R^{b}), —SO₂R^{a}, —(CH₂)₀₋₂—CO₂R^{a}, —(CH₂)₀₋₂—N(R^{a})CO₂R^{b}, —NO₂, —SR^{a}, —N(R^{a}R^{b}) and phenyl;
and all other variables are as defined in this class.

Another sub-class of the preceding class of compounds of the present invention includes a compound of Formula (I-1), or a pharmaceutically acceptable salt thereof, wherein:

R¹ is —C₁₋₃ alkyl;

### R⁴ is H or —R^{j};

### R¹ is:

(i) phenyl, which is optionally substituted with from 1 to 3 substituents each of which is independently —C₁₋₄ alkyl, —C₁₋₄ alkyl-OH, —C₁₋₄ alkyl-O—C₁₋₄ alkyl, —C₁₋₄ alkyl-O—C₁₋₄ haloalkyl, —C₁₋₄ alkyl-N(R^{a}R^{b}), —C₁₋₄ alkyl-C(═O)N(R^{a}R^{b}), —C₁₋₄ alkyl-C(═O)R^{a}, —C₁₋₄ alkyl-CO₂R^{c}, —C₁₋₄ alkyl-S(O)ₙR^{e}, —O—C₁₋₄ alkyl, —C₁₋₄ haloalkyl, —O—C₁₋₄ haloalkyl, —OH, halo, —N(R^{a}R^{b}), —C(═O)N(R^{a}R^{b}), —C(═O)R^{a}, —CO₂R^{c}, —SH, —S(O)ₙR^{e}, or —SO₂N(R^{a}R^{b}); or
(ii) a saturated heterocyclic ring selected from the group consisting of piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, thiadiazepanyl, dithiazepanyl, azepanyl, diazepanyl, thiadiazinanyl, and dioxanyl; wherein the saturated heterocyclic ring is:
   (a) optionally substituted with from 1 to 4 substituents each of which is independently halogen, —C₁₋₄ alkyl, —C₁₋₄ haloalkyl, —O—C₁₋₄ alkyl, —O—C₁₋₄ haloalkyl, or oxo; and
   (b) optionally mono-substituted with phenyl or HetA; wherein HetA is a heteroaromatic ring selected from the group consisting of pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl; wherein the heteroaromatic ring is optionally substituted with from 1 to 3 substituents each of which is independently —C₁₋₄ alkyl, —C₁₋₄ haloalkyl, —O—C₁₋₄ alkyl, —O—C₁₋₄ haloalkyl, or oxo;
      ring A is oxadiazolyl, triazolyl, thiadazolyl, oxazolyl, tetrazolyl or pyrimidinyl;
      Q is —C₁₋₃ alkylene; and
      R⁵ is phenyl, wherein the phenyl is optionally substituted with from 1 to 3 substituents each of which is independently fluoro, bromo, chloro, —OH, —C₁₋₄ alkyl, —C₁₋₄ fluoroalkyl, —O—C₁₋₄ alkyl, —O—C₁₋₄ fluoroalkyl, —(CH₂)₁₋₂—N(R^{a}R^{b}), —SO₂R^{a}, —(CH₂)₀₋₂—CO₂R^{a}, —(CH₂)₀₋₂—N(R^{a})CO₂R^{b}, —NO₂, —SR^{a}, —N(R^{a}R^{b}) and phenyl;
      and all other variables are as defined in this class.

In an aspect of the preceding sub-class, R^{j} is phenyl, which is optionally substituted with from 1 to 3 substituents each of which is independently —C₁₋₄ alkyl, —C₁₋₄ alkyl-OH, —C₁₋₄ alkyl-O—C₁₋₄ alkyl, —C₁₋₄ alkyl-O—C₁₋₄ haloalkyl, —C₁₋₄ alkyl-N(R^{a}R^{b}), —C₁₋₄ alkyl-C(═O)N(R^{a}R^{b}), —C₁₋₄ alkyl-C(═O)R^{a}, —C₁₋₄ alkyl-CO₂R^{d}, —C₁₋₄ alkyl-S(O)ₙR^{d}, —O—C₁₋₄ alkyl, —C₁₋₄ haloalkyl, —O—C₁₋₄ haloalkyl, —OH, halo, —N(R^{a}R^{b}), —C(═O)N(R^{a}R^{b}), —C(═O)R^{a}, —CO₂R^{c}, —SH, —S(O)ₙR^{d}, or —SO₂N(R^{a}R^{b}). In a feature of the preceding sub-class, R⁴ is H or phenyl.

In another aspect of the preceding sub-class, R⁵ is phenyl, wherein the phenyl is optionally substituted with from 1 to 3 substituents each of which is independently fluoro, bromo, chloro, —OH, —C₁₋₄ alkyl, —C₁₋₄ fluoroalkyl, —O—C₁₋₁₄ alkyl, —O—C₁₋₄ fluoroalkyl, —(CH₂)₁₋₂—N(R^{a}R^{b}), —SO₂R^{a}, —(CH₂)₀₋₂—CO₂R^{a}, —(CH₂)₀₋₂—N(R^{a})CO₂R^{b}, —NO₂, or phenyl.

In yet another aspect of the preceding sub-class, R¹ is methyl; ring A is 1,2,4-oxadiazolyl, 1,3,4-oxadiazolyl, 1,2,4-triazolyl, 1,3,4-thiadazolyl, 1,3-oxazol-2-yl, 2-tetrazol-5-yl, 1-tetrazol-5-yl or 4-pyrimidinyl; and Q is —CH₂—.

It is to be understood that additional embodiments of the present invention include, but are not limited to, compounds of Formula (I) wherein each of two or three or more of the same variable is independently defined in accordance with its definition in one of the embodiments or an aspect thereof as set forth above, or in accordance with its definition in one of the foregoing classes set forth above or a sub-class or feature thereof.

Furthermore, the compounds of the present invention can exist as tautomers such as the following:

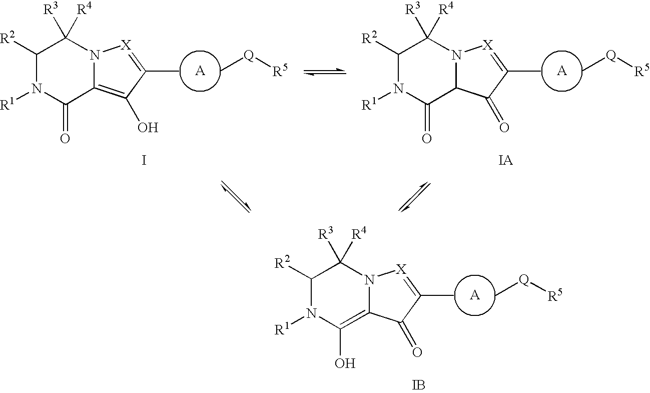

It is to be understood for the purposes of the present invention that a reference herein to a compound of Formula (I) is a reference to compound I per se, or to any one of its tautomers per se (e.g., IA or IB), or to mixtures of two or more of the tautomers (e.g., two or more of I, IA, and IB).

Another embodiment of the present invention is a compound, or a pharmaceutically acceptable salt thereof, selected from the group consisting of the compounds set forth in Examples 1 to 5 below.

Other embodiments of the present invention include the following:

(a) A pharmaceutical composition comprising an effective amount of a compound of Formula (I) and a pharmaceutically acceptable carrier.

(b) A pharmaceutical composition which comprises the product prepared by combining (e.g., mixing) an effective amount of a compound of Formula (I) and a pharmaceutically acceptable carrier.

(c) The pharmaceutical composition of (a) or (b), further comprising an effective amount of an HIV infection/AIDS treatment agent selected from the group consisting of HIV/AIDS antiviral agents, immunomodulators, and anti-infective agents.

(d) The pharmaceutical composition of (c), wherein the HIV infection/AIDS treatment agent is an antiviral selected from the group consisting of HIV protease inhibitors, non-nucleoside HIV reverse transcriptase inhibitors, and nucleoside HIV reverse transcriptase inhibitors.

(e) A pharmaceutical combination which is (i) a compound of Formula (I) and (ii) an HIV infection/AIDS treatment agent selected from the group consisting of V/AIDS antiviral agents, immunomodulators, and anti-infective agents; wherein the compound of Formula (I) and the HIV infection/AIDS treatment agent are each employed in an amount that renders the combination effective for inhibiting HIV integrase, for treating or preventing infection by HIV, or for preventing, treating or delaying the onset of AIDS.

(f) The combination of (e), wherein the HIV infection/AIDS treatment agent is an antiviral selected from the group consisting of HIV protease inhibitors, non-nucleoside HIV reverse transcriptase inhibitors and nucleoside HIV reverse transcriptase inhibitors.

(g) A method of inhibiting HIV integrase in a subject in need thereof which comprises administering to the subject an effective amount of a compound of Formula (I).

(h) A method of preventing or treating infection by HIV in a subject in need thereof which comprises administering to the subject an effective amount of a compound of Formula (I).

(i) The method of (h), wherein the compound of Formula (I) is administered in combination with an effective amount of at least one antiviral selected from the group consisting of HIV protease inhibitors, non-nucleoside HIV reverse transcriptase inhibitors, and nucleoside HIV reverse transcriptase inhibitors.

(j) A method of preventing, treating or delaying the onset of AIDS in a subject in need thereof which comprises administering to the subject an effective amount of a compound of Formula (I).

(k) The method of (j), wherein the compound is administered in combination with an effective amount of at least one antiviral selected from the group consisting of HIV protease inhibitors, non-nucleoside HIV reverse transcriptase inhibitors, and nucleoside HIV reverse transcriptase inhibitors

(l) A method of inhibiting HIV integrase in a subject in need thereof which comprises administering to the subject the pharmaceutical composition of (a), (b), (c) or (d) or the combination of (e) or (f).

(m) A method of preventing or treating infection by HIV in a subject in need thereof which comprises administering to the subject the pharmaceutical composition of (a), (b), (c) or (d) or the combination of (e) or (f).

(n) A method of preventing, treating or delaying the onset of AIDS in a subject in need thereof which comprises administering to the subject the pharmaceutical composition of (a), (b), (c) or (d) or the combination of (e) or (f).

The present invention also includes a compound of the present invention (i) for use in, (ii) for use as a medicament for, or (iii) for use in the preparation of a medicament for: (a) inhibiting HIV integrase, (b) preventing or treating infection by HIV, or (c) preventing, treating or delaying the onset of AIDS. In these uses, the compounds of the present invention can optionally be employed in combination with one or more HIV/AIDS treatment agents selected from HIV/AIDS antiviral agents, anti-infective agents, and immunomodulators.

Additional embodiments of the invention include the pharmaceutical compositions, combinations and methods set forth in (a)-(n) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt.

As used herein, the term “alkyl” refers to any linear or branched chain allyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “alkylene” refers to any linear or branched chain alkylene group (or alternatively “alkanediyl”) having a number of carbon atoms in the specified range. Thus, for example, “—C₁₋₆ alkylene-” refers to any of the C₁ to C₆ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Also of interest is the alkylene —CH(CH₃)—.

The terms “cycloalkyl” refers to any cyclic ring of an alkane having a number of carbon atoms in the specified range. Thus, for example, “C₃₋₈ cycloalkyl” (or “C₃-C₈ cycloalkyl”) refers to cyclopropyl, cyclobutyl, cyclopentyl, cyclohexyl, cycloheptyl, and cyclooctyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or 1). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.).

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, and so forth. The heteroatoms in any of the heterocyclic rings of the present compounds include any oxidized form of nitrogen (e.g., N′—O⁻) and sulfur (e.g., S(O) and SO₂) and the quaternized form of any basic nitrogen.

The term “oxo” means a divalent oxygen substituent; i.e., ═O.

Representative examples of saturated heterocyclic rings include piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, tetrahydrothienyl, pyrazolidinyl, hexahydropyrimidinyl, thiazinanyl (e.g., 1,2-thiazinanyl

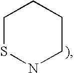

thiazepanyl, thiadiazepanyl, dithiazepanyl, azepanyl (i.e.,

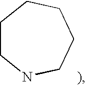

diazepanyl, thiadiazinanyl (e.g., 1,2,6-thiadiazinanyl

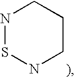

and dioxanyl. Representative examples of mono-unsaturated rings are the same as the saturated rings listed in the preceding sentence except that each ring contains a double bond.

Representative examples of 5- or 6-membered saturated or unsaturated nonaromatic (i.e., partially saturated) heterocyclic rings include piperidinyl, pyrazolidinyl, imidazolidinyl, piperazinyl, piperidinyl, and hexahydropyrimidinyl.

Representative examples of heteroaromatic rings of the present invention include pyridyl, pyrrolyl, pyrazinyl, pyrimidinyl, pyridazinyl, triazinyl, thienyl (or thiophenyl), thiazolyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl.

Representative examples of fused bicyclic heterocycles useful in the present invention include benzotriazolyl, indolyl, isoindolyl, indazolyl, indolinyl, isoindolinyl, quinoxalinyl, quinazolinyl, cinnolinyl, chromanyl, isochromanyl, tetrahydroquinolinyl, quinolinyl, tetrahydroisoquinolinyl, isoquinolinyl, 2,3-dihydrobenzofuranyl, 2,3-dihydrobenzo-1,4-dioxinyl (i.e.,

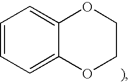

and benzo-1,3-dioxolyl (i.e.,

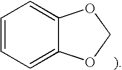

When any variable (e.g., R^{a}, R^{b}, R^{c}, R^{d}, or R^{j}) occurs more than one time in any constituent or in Formula (I) or in any other formula depicting and describing compounds of the invention, its definition on each occurrence is independent of its definition at every other occurrence. Also, combinations of substituents and/or variables are permissible only if such combinations result in stable compounds.

The term “substituted” (e.g., as in “is optionally substituted with from 1 to 5 substituents . . . ”) includes mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., aryl, a heteroaromatic ring, or a saturated heterocyclic ring) provided such ring substitution is chemically allowed and results in a stable compound.

The symbol [symbol] in front of an open bond in the structural formula of a group marks the point of attachment of the group to the rest of the molecule.

The compounds of the present invention may have asymmetric centers and may occur, except when specifically noted, as mixtures of stereoisomers or as individual diastereomers, or enantiomers, with all isomeric forms being included in the present invention.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain of the compounds of the present invention can have asymmetric centers and can occur as mixtures of stereoisomers, or as individual diastereomers, or enantiomers. All isomeric forms of these compounds, whether isolated or in mixtures, are within the scope of the present invention.

The compounds of the present inventions are useful in the inhibition of HIV integrase (e.g., HIV-1 integrase), the prevention or treatment of infection by human immunodeficiency virus (HIV) and the prevention, treatment or the delay in the onset of consequent pathological conditions such as AIDS. Preventing AIDS, treating AIDS, delaying the onset of AIDS, or preventing or treating infection by UV is defined as including, but not limited to, treatment of a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by such means as blood transfusion, exchange of body fluids, bites, accidental needle stick, or exposure to patient blood during surgery.

The compounds of this invention are useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention are commercial products to be sold for these purposes.

The compounds of the present invention may be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Many of the compounds of the invention carry an acidic moiety, in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of the invention mean providing the compound or a prodrug of the compound to the individual in need of treatment. When a compound of the invention or a prodrug thereof is provided in combination with one or more other active agents (e.g., antiviral agents useful for treating UV infection or AIDS), “administration” and its variants are each understood to include concurrent and sequential provision of the compound or prodrug and other agents.

As used herein, the term “composition” is intended to encompass a product comprising the specified ingredients, as well as any product which results, directly or indirectly, from combining the specified ingredients. The term “compound” unless otherwise indicated is intended to encompass both the compound itself and pharmaceutically acceptable salt and/or hydrates thereof.

By “pharmaceutically acceptable” is meant that the ingredients of the pharmaceutical composition must be compatible with each other and not deleterious to the recipient thereof.

The term “subject” (alternatively referred to herein as “patient”) as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV integrase and thereby elicit the response being sought (i.e., an “inhibition effective amount”). When the active compound (i.e., active ingredient) is administered as the salt, references to the amount of active ingredient are to the free acid or free base form of the compound.

For the purpose of inhibiting HIV integrase, preventing or treating UV infection or preventing, treating or delaying the onset of AIDS, the compounds of the present invention, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions of the present invention and of ingredients suitable for use in said compositions is provided in Remington's Pharmaceutical Sciences, 18^{th} edition, edited by A. R. Gennaro, Mack Publishing Co., 1990.

The compounds of this invention can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to use of the HIV integrase inhibitor compounds of the present invention with one or more agents useful in the treatment of HIV infection or AIDS. For example, the compounds of this invention may be effectively administered, whether at periods of pre-exposure and/or post-exposure, in combination with effective amounts of one or more HIV/AIDS antivirals, immunomodulators, antiinfectives, or vaccines useful for treating HIV infection or AIDS, such as those disclosed in Table 1 of WO 01/38332 or in the Table in WO 02/30930. Suitable HIV/AIDS antivirals for use in combination with the compounds of the present invention include, for example, HIV protease inhibitors (e.g., indinavir, atazanavir, lopinavir optionally with ritonavir, saquinavir, or nelfinavir), nucleoside HIV reverse transcriptase inhibitors (e.g., abacavir, lamivudine (3TC), zidovudine (AZT), or tenofovir), and non-nucleoside HIV reverse transcriptase inhibitors (e.g., efavirenz or nevirapine). It will be understood that the scope of combinations of the compounds of this invention with HIV/AIDS antivirals, immunomodulators, anti-infectives or vaccines is not limited to the foregoing substances or to the list in the above-referenced Tables in WO 01/38332 and WO 02/30930, but includes in principle any combination with any pharmaceutical composition useful for the treatment of AIDS. The HIV/AIDS antivirals and other agents will typically be employed in these combinations in their conventional dosage ranges and regimens as reported in the art, including, for example, the dosages described in the Physicians' Desk Reference, 57^{th} edition, Thomson P D R, 2003. The dosage ranges for a compound of the invention in these combinations are the same as those set forth above.

The compounds of the present invention can be readily prepared according to the following reaction schemes and examples, or modifications thereof, using readily available starting materials, reagents and conventional synthesis procedures. For examples, various scaffolds of the present compounds may be assembled following the teachings of international patent publication WO2004/101512, WO2004/047725, and U.S. patent publication US2005/0025774. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following examples.

A general method for the preparation of compounds of the present invention embraced by Formula I is shown in Scheme 1.

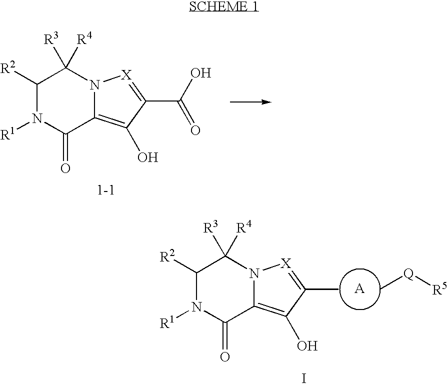

General method for the preparation of alkyl 8-hydroxy-1-oxo-1,2,3,4-tetrahydropyrrolopyrazine-7-carboxylic acid 1-1[X═C(R⁷)] and 8-hydroxy-1-oxo-1,2,3,4-tetrahydropyrazolopyrazine-7-carboxylic acid 1-1 (X═N) are outlined in Schemes 2 and 3, respectively. These key intermediates are then transformed to the present invention embraced by Formula I as described in Schemes 4, 5, 6, and 7.

Treatment of the amine-protected piperazin-2-one 2-1 (Scheme 2) with a deprotonating reagent (e.g., Li or Na bis(trimethylsilyl)amide or Na hydride) at low temperature (e.g., from about 0 to about 25° C.) in an anhydrous non-protic solvent (e.g., DMF or THF), followed by addition of an alkylating reagent provided the intermediate 2-2. Removal of the amino protecting group provided piperazin-2-one 2-3. Compound 2-1 can be prepared using methods described in Choi et al., J. Med. Chem. 1999, 3647; Najman-Bronzewska et al., Pharmazie 1997, 198; Fryer et al., J. Org. Chem. 1991, 3715, Dinsmore et al, Organic Prep. & Procedures International, 2002, 369, or routine variations thereof. An alternative method for preparing piperazin-2-one 2-3 is described in Bernotas et al., Tetrahedron Lett. 1996, 7339; Saari et al., J. Med. Chem. 1990, 2590; Sugihara et al., J. Med. Chem. 1998, 489, Dinsmore et al, Organic Prep. & Procedures International. 2002, 369, or routine variations thereof. The protection and deprotection of the amine in the piperazin-2-one can be accomplished using conventional amine protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 1991. Piperazin-2-one 2-3 is treated with dialkylalkoxymethylenemalonate 2-4. The resultant adduct 2-5 is treated with a deprotonating agent (e.g., Li or Na bis(trimethylsilyl)amide or Na hydride) in an anhydrous non-protic solvent (e.g., DMF or THF) at 0° C. to 80° C. to give alkyl 8-hydroxy-1-oxo-1,2,3,4-tetrahydropyrrolopyrazine-7-carboxylate 2-6. The hydroxy group on the pyrrolopyrazine carboxylate 2-6 was protected as ether (e.g., methyl or benzyl ether) and the carboxylate ester is hydrolyzed to give the 7-carboxylic acid 1-1[X═C(R⁷)]. Some of the suitable dialkylalkoxymethylenemalonates 2-4 are commercially available (e.g., diethylethoxymethylenemalonate or dimethylmethoxymethylenemalonate). Others can be obtained by preparative methods known in the art; e.g., heterocyclylalkyloxy-methylene malonates can be prepared by the method described in Boger et al., J. Org. Chem 1988, 3408, or routine variations thereof.

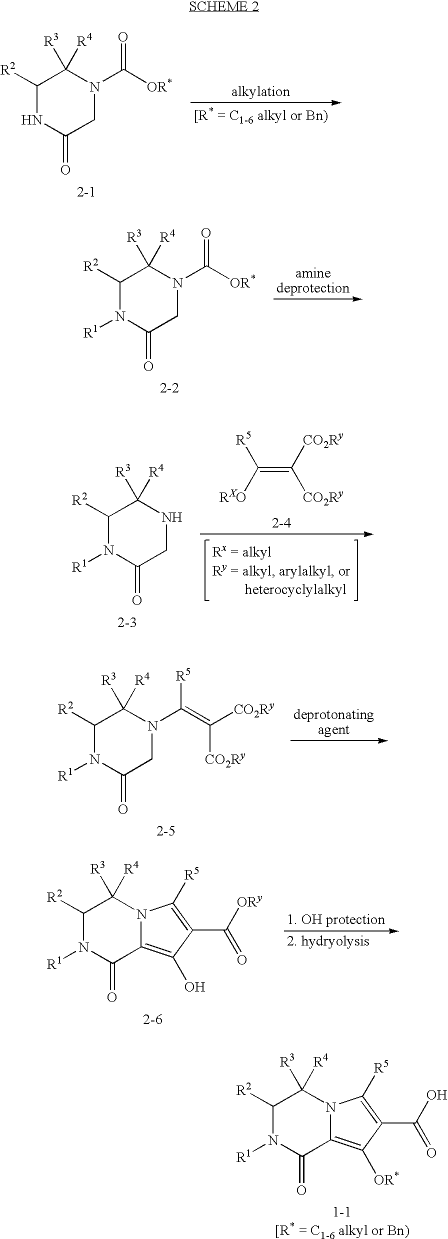

Diester pyrazole 3-1 (Rodriguez-Franco, et al. Tetrahedron 1999, 55, 2763-72) is selectively converted to mono acid 3-2 using dimethylhydrazine (Scheme 3). Coupling acid 3-2 to an amino alcohol derivative using standard peptide coupling conditions gives amide 3-3. Ring closure of 3-3 to give 3-4 can be accomplished using Mitsunobu conditions using reagents such as diethyl azodicarboxylate and triphenylphosphine. Hydrolysis of ester 3-4 provides the intermediate carboxylic acid 1-1 (X═N) for further functionalization.

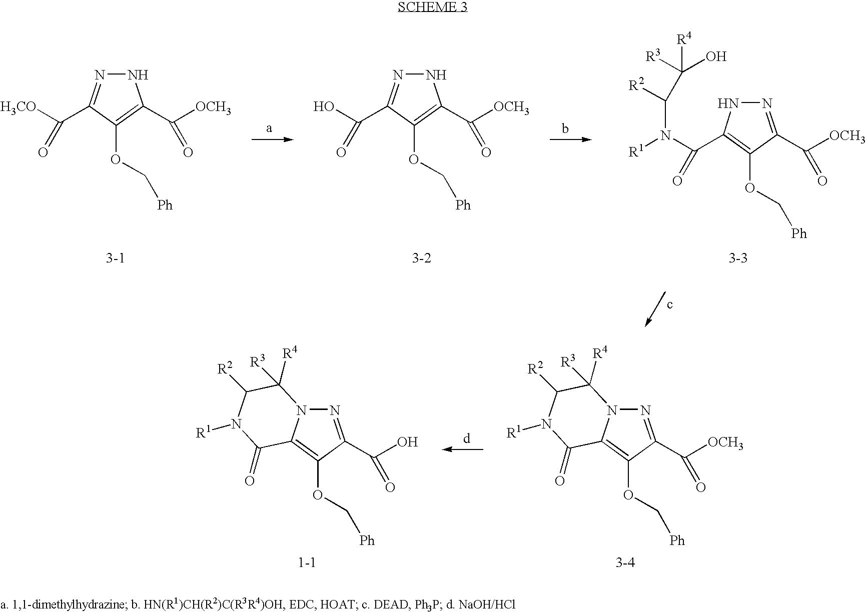

The key intermediates 1-1[X═C(R⁷) and N] are transformed to the present invention embraced by Formula I as described in following schemes (4, 5, 6, and 7).

Acid 1-1[X═C(R⁷) and N] is coupled with acyl hydrazide under standard amide coupling conditions (such as BOP, PyBOP, EDC/HOBt, see Brown, B. J. Synlett 2000, 131) to provide diacyl hydrazide 4-1 (Scheme 4). Alternatively, acid 1-1 is first converted to acyl hydrazide 4-2, which will then be treated with appropriate acid chloride in the presence of base to form diacyl hydrazide 4-1. Compound 4-2 may also be accessed from esters 2-6 and 3-4 by heating with hydrazine.

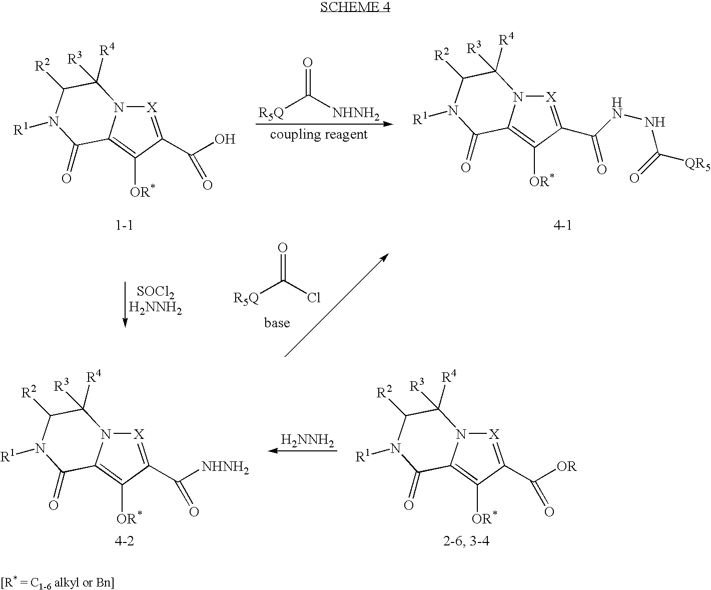

Treatment of diacyl hydrazide 4-1 with thio-dehydrating reagent (such as Lawesson's reagent, P₂S₅, see Kling, A. Bioorganic & Medicinal Chemistry 2003, 1319) provides 1,3,4-thiodiazole 5-1 (Scheme 5). Removal of hydroxy protecting group provides compound 5-2 of the present invention.

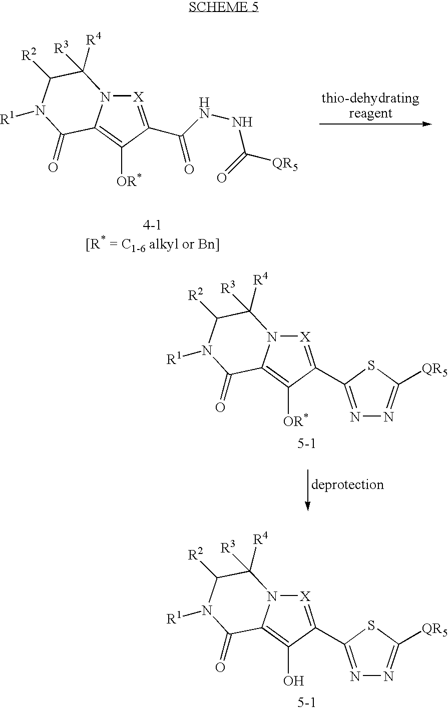

Similarly, treatment of diacyl hydrazide 4-1 with a dehydrating reagent (such as Burgess's reagent, POCl₃, SOCl₂, see Brain, C. T. Tetrahedron Lett. 1999, 3275) provides the intermediate 1,3,4-oxadiazole 6-1 (Scheme 6). Removal of the protecting group at the phenolic hydroxy group in compound 6-1 provides 6-2 of this invention.

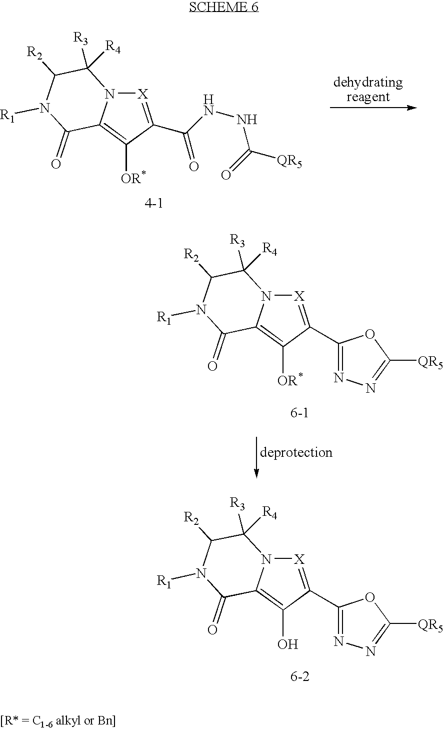

Heating compound 6-1 with benzylamine provides 1,2,4-triazole 7-1 (Scheme 7) (Kakefuda, A. Bioorganic & Medicinal Chemistry 2002, 1905). Cleavage of N-benzyl group on the resultant triazole with catalytic hydrogenolysis affords compound 7-2. Final removal of the protecting group at the phenolic hydroxy group furnishes compound 7-3 of this invention.

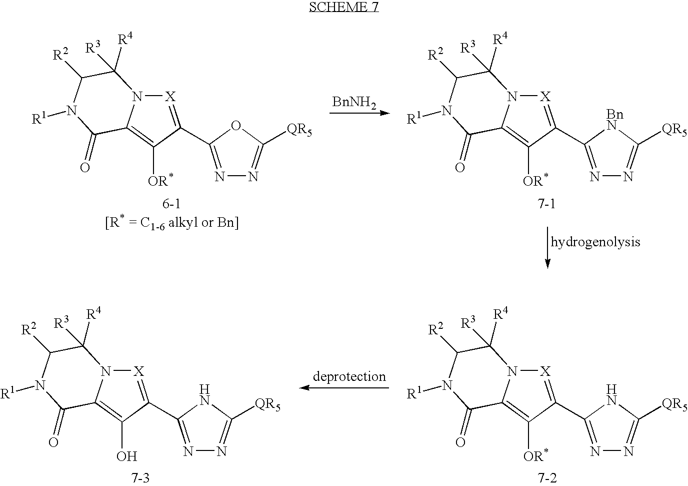

The inhibitory activity of the compounds of the present invention may be measured by assays known in the art. Representative compounds of the present invention exhibit inhibition of strand transfer activity in the HIV integrase assay as described in WO 02/30930 for recombinant integrase. Preferred compounds have IC₅₀ values of ≦1 micromolar in this integrase assay. Representative compounds of the present invention exhibit inhibition of HIV replication in the assay as described in Joyce, J. G., et al., J. Biol. Chem., 2002, 277, 45811, Hazuda, D. J. et al., Science, 2000, 287, 646, and Kimpton, J. et al, J. Virol. 1992, 66, 2232 for measuring the inhibition of acute HIV infection with HeLa P4-2 cells in a single cycle infectivity assay. Preferred compounds have IC₅₀ values of <35 micromolar in this HIV replication assay.

The following examples serve only to illustrate the invention and its practice. The examples are not to be construed as limitations on the scope or spirit of the invention.

### Example 1

### 7-[5-(4-Fluorobenzyl)-1,3,4-thiadiazol-2-yl]-8-hydroxy-2-methyl-3,4-dihydropyrrolo[1,2-a]pyrazin-1(2H)-one

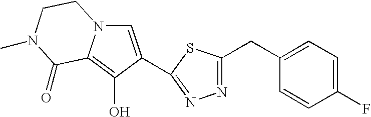

### Step 1: N²-Benzyloxycarbonyl-N¹-(2,2-dimethoxyethyl)-N¹-methylglycinamide

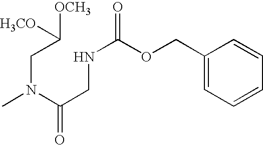

A solution of N-(2,2-dimethoxyethyl)-N-methylamine (760 g, 6.38 mmol), N—CBZ-glycine (1337.6 g, 6.39 mol), 1-(3-dimethylaminopropyl)-3-ethylcarbodiimide hydrochloride (1225.8 g, 6.39 mol; EDC), and 1-hydroxybenzotriazole hydrate (107.7 g, 0.70 mol; HOBT), and N,N-diisopropylethylamine (172 mL) in anhydrous DMF (12 L) was stirred at room temperature overnight. The reaction mixture was diluted with water (24 L) and extracted with dichloromethane (3×10 L). The organic extracts were combined, dried over anhydrous magnesium sulfate, filtered, and concentrated under vacuum to provide the title compound. ES MS M+1=311.

### Step 2: 4-Benzyloxycarbonyl-1-methyl-3,4-dihydropyrazin-2(1H)-one

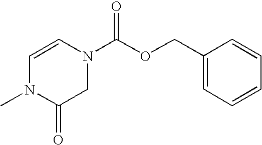

A solution of N²-benzyloxycarbonyl-N¹-(2,2-dimethoxyethyl)-N¹-methylglycinamide (1.9 Kg, 6.1 mol) and p-toluenesulfonic acid monohydrate (270 g) in toluene (29.4 L) was stirred at 80° C. for 4 hrs. The resultant reaction mixture was cooled to room temperature, washed with water (4×2 L), dried over anhydrous magnesium sulfate, filtered, and concentrated under vacuum. The residual solid was subjected to column chromatography on silica gel eluting with heptane-ethyl acetate. Concentration of appropriate fractions provided the cyclization product as off-white solid. ¹H NMR (400 MHz, CDCl₃) δ 7.37 (br s, 5H), 6.44 (d, J=6.0 Hz, ½H), 6.32 (d, J=6.0 Hz, ½H), 5.53 (d, J=6.0 Hz, ½H), 5.42 (d, J=6.0 Hz, ½H), 5.21 (s, 2H), 4.31 (s, 2H), 3.08 (s, 3H). ES MS M+1=247.

### Step 3: 1-methylpiperazin-2-one

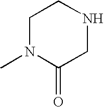

A mixture of 4-benzyloxycarbonyl-1-methyl-3,4-dihydropyrazin-2(1H)-one (510 g, 2.1 mol) and 10% Pt/C (40 g) in ethanol (12 L) was stirred under an atmosphere of hydrogen (1 atm) at room temperature overnight. Pearlmans catalyst (50 g; 20% Pd(OH)₂ on C) was added and stirred under an atmosphere of hydrogen gas for additional 24 hours. The product mixture was filtered through a pad of Celite, and concentrated under vacuum to provide 1-methylpiperazin-2-one. ¹H NMR (400 MHz, CDCl₃) δ 3.52 (s, 2H), 3.32 (t, J=5.7 Hz, 2H) 3.09 (t, J=5.7 Hz, 2H), 2.97 (s, 3H).

### Step 4: Ethyl 8-hydroxy-2-methyl-1-oxo-1,2,3,4-tetrahydropyrrolo[1,2-a]-pyrazine-7-carboxylate

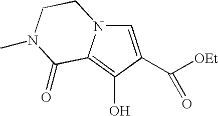

A mixture of 1-methylpiperazin-2-one (183 g, 1.6 mol) and diethyl ethoxymethylenemalonate (346 g, 1.6 mol) in toluene (12 L) was heated at 100° C. overnight. The resultant mixture was concentrated under vacuum. The residue was dissolved in anhydrous THF (8 L), brought to reflux under an atmosphere of nitrogen, and treated with a solution of lithium bis(trimethylsilyl)amide in THF (1 M, 1.05 eq). The reaction mixture was allowed to cool to room temperature and concentrated under vacuum. The residue was partitioned between methylene chloride and dilute aqueous HCl. The organic extract was washed with brine, dried over anhydrous magnesium sulfate, filtered, and concentrated under vacuum. The residue was triturated with ethyl acetate, cooled to −20° C., and the solid precipitated was filtered to provide the title compound. ¹H NMR (400 MHz, DMSO-d₆) δ 8.50 (s, 1H), 7.33 (s, 1H), 4.18 (q, J=7.1 Hz, 2H), 4.11 (t, J=5.5 Hz, 2H), 3.59 (t, J=5.5 Hz, 2H), 2.92 (s, 3H), 1.24 (t, J=7.1 Hz, 3H). ES MS M+1=239.

### Step 5: Ethyl 8-methoxy-2-methyl-1-oxo-1,2,3,4-tetrahydropyrrolo[1,2-a]pyrazine-7-carboxylate

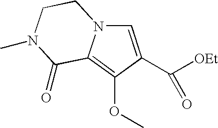

A mixture of ethyl 8-hydroxy-2-methyl-1-oxo-1,2,3,4-tetrahydropyrrolo[1,2-a]-pyrazine-7-carboxylate (11.1 g, 46.8 mmol), potassium carbonate (25.8 g, 187.0 mmol; 325 mesh), and iodomethane (3.50 mL, 56.1 mmol) in DMF (50 mL) was stirred at room temperature overnight. The mixture was filtered and the filtrate concentrated under vacuum. The residue was partitioned between chloroform and dilute aqueous HCl. The organic extract was washed with brine, dried over sodium sulfate, filtered, and concentrated under vacuum. The residue was subjected to column chromatography on silica gel eluted with ethyl acetate to give titled material. ¹H NMR (400 MHz, CDCl₃) δ 7.12 (s, 1H), 4.28 (q, J=7.1 Hz, 2H), 4.09 (t, J=5.7 Hz, 2H), 4.03 (s, 3H), 3.62 (t, J=5.7 Hz, 2H), 3.10 (s, 3H), 1.34 (t, J=7.1 Hz, 3H).

### Step 6: 8-Methoxy-2-methyl-1-oxo-1,2,3,4-tetrahydropyrrolo[1,2-a]pyrazine-7-carboxylic acid

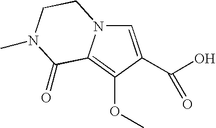

A solution of ethyl 8-methoxy-2-methyl-1-oxo-1,2,3,4-tetrahydropyrrolo[1,2-a]pyrazine-7-carboxylate (1.0 g, 4.0 mmol) and 1N aqueous sodium hydroxide (10 mL) in ethanol (50 mL) was stirred at room temperature for 4 days. The mixture was acidified with 1N HCl (15 mL) and concentrated under vacuum. The resulting solid was collected by filtration to give the titled material.

¹H NMR (400 MHz, DMSO-d₆) δ 11.93 (br s, 1H), 7.34 (s, 1H), 4.13-4.08 (m, 2H), 3.79 (s, 3H), 3.59 (t, J=5.8 Hz, 2H), 2.94 (s, 3H). ES MS M+1=225.

### Step 7: N′-[(4-Fluorophenyl)acetyl]-8-methoxy-2-methyl-1-oxo-1,2,3,4-tetrahydropyrrolo[1,2-a]pyrazine-7-carbohydrazide

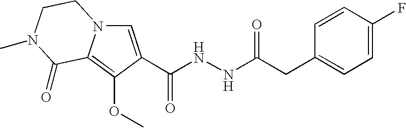

A mixture of 8-methoxy-2-methyl-1-oxo-1,2,3,4-tetrahydropyrrolo[1,2-a]pyrazine-7-carboxylic acid (0.5 g, 2.2 mmol), 2-(4-fluorophenyl)acetohydrazide (0.5 g, 2.7 mmol; prepared from 4-fluorophenyl acetyl chloride and hydrazine in a manner similar to that described in J. Heterocyclic Chemistry, 1977, 14, 1123) and (1H-1,2,3-benzotriazol-1-yloxy)[tris(dimethylamino)]phosphonium hexafluorophosphate (1.5 g, 3.3 mmol), and N,N-diisopropylethylamine (1.56 mL, 8.9 mmol) in DMF (20 mL) was stirred at room temperature overnight. The product mixture was concentrated under vacuum. The residue was dissolved in chloroform (100 mL) and washed with brine (3×50 mL). The organic extract was washed dried over sodium sulfate, filtered, and concentrated under vacuum. The residue was subjected to column chromatography on silica gel eluted with methanol to give titled material. ¹H NMR (400 MHz, CDCl₃) δ 9.55 (s, 1H), 8.36 (s, 1H), 7.33-7.29 (m, 2H), 7.16 (s, 1H), 7.05 (t, J=8.7 Hz, 2H), 4.20 (s, 3H), 4.10 (t, J=5.3 Hz, 2H), 3.66-3.63 (m, 4H), 3.11 (s, 3H).

### Step 8: 7-[5-(4-Fluorobenzyl)-1,3,4-thiadiazol-2-yl]-8-methoxy-2-methyl-3,4-dihydropyrrolo[1,2-a]pyrazin-1(2H)-one

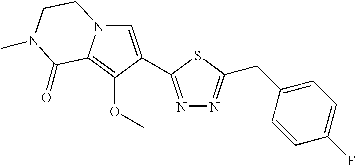

To a boiling solution of N′-[(4-fluorophenyl)acetyl]-8-methoxy-2-methyl-1-oxo-1,2,3,4-tetrahydropyrrolo[1,2-a]pyrazine-7-carbohydrazide (0.2 g, 0.5 mmol) in THF (30 mL), Lawesson's reagent (0.6 g, 1.6 mmol) was added in portions. The reaction mixture was heated under reflux for 2 hours. The product mixture was concentrated under vacuum. The residue was partitioned between dichloromethane and brine. The organic extract was dried over sodium sulfate, filtered, and concentrated under vacuum. The residue was subjected to column chromatography on silica gel eluted with methanol to give titled material. ¹H NMR (400 MHz, CDCl₃) δ 7.35 (s, 1H), 7.30 (dd, J=8.4, 5.3 Hz, 2H), 7.03 (t, J=8.6 Hz, 2H), 4.40 (s, 2H), 4.15 (t, J=5.7 Hz, 2H), 4.06 (s, 3H), 3.66 (t, J=5.7 Hz, 2H), 3.11 (s, 3H).

### Step 9: 7-[5-(4-Fluorobenzyl)-1,3,4-thiadiazol-2-yl]-8-hydroxy-2-methyl-3,4-dihydropyrrolo[1,2-a]pyrazin-1(2H)-one

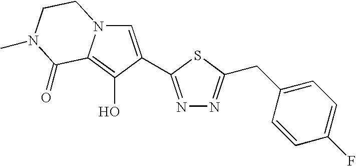

To a solution of 7-[5-(4-fluorobenzyl)-1,3,4-thiadiazol-2-yl]-8-methoxy-2-methyl-3,4-dihydropyrrolo[1,2-a]pyrazin-1(2H)-one (0.06 g, 0.16 mmol) in dichloromethane (10 mL) at room temperature, a solution of boron tribromide in dichloromethane (0.3 mL; IM) was added. The reaction mixture was stirred at room temperature for 4 hours and concentrated under vacuum. The residue was dissolved in methanol, stirred at room temperature for 10 minutes, and concentrated under vacuum. This was repeated twice. The residue was subjected HPLC purification on C-18 reverse stationary phase eluted with a gradient of water in acetonitrile with 0.1% TFA. Collection and lyophilization of appropriate fraction provided the titled compound. ¹H NMR (400 MHz, DMSO-d₆) δ 7.50 (s, 1H), 7.41 (dd, J=8.6, 5.9 Hz, 2H), 7.18 (t, J=8.7 Hz, 2H), 4.43 (s, 2H), 4.15 (t, J=5.5 Hz, 2H), 3.61 (t, J=5.5 Hz, 2H), 2.94 (s, 3H). ES MS M+1=359

### Example 2

### 7-[5-(4-Fluorobenzyl)-1,3,4-oxadiazol-2-yl]-8-hydroxy-2-methyl-3,4-dihydropyrrolo[1,2-a]pyrazin-1(2H)-one

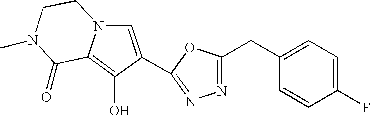

### Step 1: 7-[5-(4-Fluorobenzyl)-1,3,4-oxadiazol-2-yl]-8-methoxy-2-methyl-3,4-dihydropyrrolo[1,2-a]pyrazin-1(2H)-one

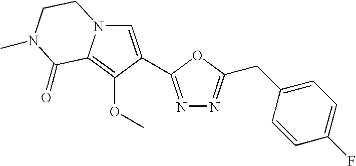

A solution of N′-[(4-fluorophenyl)acetyl]-8-methoxy-2-methyl-1-oxo-1,2,3,4-tetrahydropyrrolo[1,2-a]pyrazine-7-carbohydrazide (0.2 g, 0.5 mmol, prepared according to the procedures described in Example 1 starting with N—CBZ-glycine) and (methoxycarbonylsulfamoyl) triethylammonium hydroxide, inner salt (1.0 g, 4.3 mmol; Burgess Reagent) in THF (30 mL) was heated under reflux for 24 hours. The product mixture was concentrated under vacuum. The residue was partitioned between chloroform and brine. The organic extract was dried over sodium sulfate, filtered, and concentrated under vacuum. The residue was subjected to column chromatography on silica gel eluted with a gradient mixture of methanol in ethyl acetate. Collection and concentration of appropriate fractions afforded title material. ¹H NMR (400 MHz, CDCl₃) δ 7.33 (dd, J=8.6, 5.3 Hz, 2H), 7.15 (s, 1H), 7.03 (t, J=8.7 Hz, 2H), 4.23 (s, 2H), 4.13 (t, J=5.7 Hz, 2H), 4.02 (s, 3H), 3.65 (t, J=5.7 Hz, 2H), 3.11 (s, 3H).

### Step 2: 7-[5-(4-Fluorobenzyl)-1,3,4-oxadiazol-2-yl]-8-hydroxy-2-methyl-3,4-dihiydropyrrolo[1,2-a]pyrazin-1(2H)-one

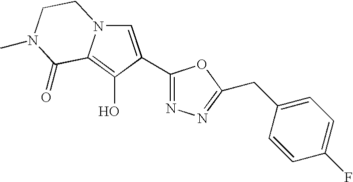

To a solution of 7-[5-(4-fluorobenzyl)-1,3,4-oxadiazol-2-yl]-8-methoxy-2-methyl-3,4-dihydropyrrolo[1,2-a]pyrazin-1(2H)-one (0.08 g, 0.22 mmol) in dichloromethane (15 mL) at room temperature, a solution of boron tribromide in dichloromethane (0.5 mL; 1M) was added. The reaction mixture was stirred at room temperature for 4 hours and concentrated under vacuum. The residue was dissolved in methanol, stirred at room temperature for 10 minutes, and concentrated under vacuum. This was repeated twice. The residue was subjected HPLC purification on C-18 reverse stationary phase eluted with a gradient of water in acetonitrile with 0.1% TFA. Collection and lyophilization of appropriate fraction provided the titled compound. ¹H NMR (400 MHz, DMSO-d₆) δ 7.43 (s, 1H), 7.38 (dd, J=8.6, 5.6 Hz, 2H), 7.19 (t, J=8.9 Hz, 2H), 4.28 (s, 2H), 4.14 (t, J=5.7 Hz, 2H), 3.62 (t, J=5.7 Hz, 2H), 2.94 (s, 3H). ES MS M+1=343.

### Example 3

### 7-[5-(4-Fluorobenzyl)-1,2,4-triazol-2-yl]-8-hydroxy-2-methyl-3,4-dihydropyrrolo[1,2-a]pyrazin-1(2H)-one

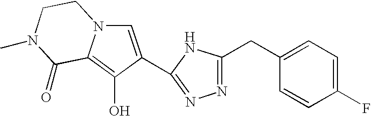

Step 1: 7-[4-Benzyl-5-(4-fluorobenzyl)-4H-1,2,4-triazol-3-yl]-8-methoxy-2-methyl-3,4-dihydropyrrolo[1,2-a]pyrazin-1(2H)-one

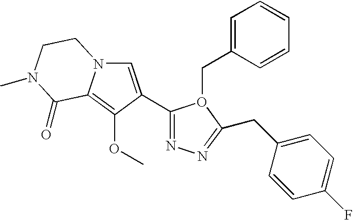

A mixture of 7-[5-(4-fluorobenzyl)-1,3,4-oxadiazol-2-yl]-8-methoxy-2-methyl-3,4-dihydropyrrolo[1,2-a]pyrazin-1(2H)-one (0.2 g, 0.5 mmol) and benzylamine (0.5 mL, 4.6 mmol) in toluene (4 mL) was heated at 170° C. for 72 hours. The mixture was concentrated under vacuum. The residue was subjected to column chromatography on silica gel eluted with 20% methanol in ethyl acetate. Collection and concentration of appropriate fractions afforded title material. ¹H NMR (400 MHz, CDCl₃) δ 7.36-7.31 (m, 2H), 7.24-7.21 (m, 2H), 7.08-7.05 (m, 1H), 6.95-6.90 (m, 3H), 6.82-6.79 (m, 2H), 5.04 (s, 2H), 4.09-4.07 (m, 2H), 3.95 (s, 2H), 3.89 (s, 1H), 3.80 (s, 3H), 3.64-3.61 (m, 2H), 3.09 (s, 3H).

### Step 2: 7-[5-(4-Fluorobenzyl)-4H-1,2,4-triazol-3-yl]-8-methoxy-2-methyl-3,4-dihydropyrrolo[1,2-a]pyrazin-1(2H)-one

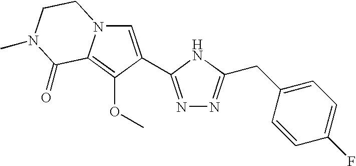

A mixture of 7-[4-benzyl-5-(4-fluorobenzyl)-4H-1,2,4-triazol-3-yl]-8-methoxy-2-methyl-3,4-dihydropyrrolo[1,2-a]pyrazin-1(2H)-one (0.2 g, 0.3 mmol) and Pearlman's catalyst (0.06 g) in ethanol (30 mL) was stirred under a balloon of hydrogen gas at room temperature overnight. The mixture was filtered through a pad of Celite. The filtrate was concentrated under vacuum to provide titled material. ¹H NMR (400 MHz, CDCl₃) δ 7.30 (dd, J=8.4, 5.7 Hz, 2H), 7.21 (s, 1H), 6.97 (t, J=8.6 Hz, 2H), 4.16 (s, 2H), 4.11 (t, J=5.7 Hz, 2H), 4.08 (s, 3H), 3.65 (t, J=5.7 Hz, 2H), 3.12 (s, 3H).

### Step 3: 7-[5-(4-Fluorobenzyl)-4H-1,2,4-triazol-3-yl]-8-hydroxy-2-methyl-3,4-dihydropyrrolo[1,2-a]pyrazin-1 (2H)-one

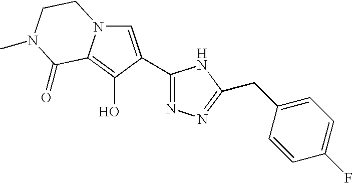

To a solution of 7-[5-(4-fluorobenzyl)-4H-1,2,4-triazol-3-yl]-8-methoxy-2-methyl-3,4-dihydropyrrolo[1,2-a]pyrazin-1(2H)-one (0.015 g, 0.042 mmol) in dichloromethane (10 mL) at room temperature, a solution of boron tribromide in dichloromethane (0.5 mL; IM) was added. The reaction mixture was stirred at room temperature for 4 hours and concentrated under vacuum. The residue was dissolved in methanol, stirred at room temperature for 10 minutes, and concentrated under vacuum. This was repeated twice. The residue was subjected HPLC purification on C-18 reverse stationary phase eluted with a gradient of water in acetonitrile with 0.1% TFA. Collection and lyophilization of appropriate fraction provided the titled compound. ¹H NMR (400 MHz, DMSO-d₆) δ 7.35 (dd, J=8.4, 5.8 Hz, 2H), 7.31 (s, 1H), 7.15 (t, J=8.8 Hz, 2H), 4.14 (t, J=5.5 Hz, 2H), 4.09 (s, 2H), 3.62 (t, J=5.5 Hz, 2H), 2.94 (s, 3H). ES MS M+1=342.

### Example 4

### (7S)-2-[5-(4-Fluorobenzyl)-1,3,4-thiadiazol-2-yl]-3-hydroxy-5-methyl-7-phenyl-6,7-dihydropyrazolo[1,5a]pyrazin-4(5H)-one

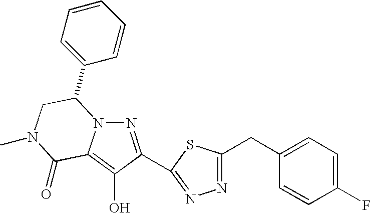

### Step 1: Methyl 4-benzyloxy-3-carboxy-1H-pyrazole-5-carboxylate

A mixture of dimethyl 4-benzyloxy-1H-pyrazole-3,5-dicarboxylate (25 g, 86 mmol) and N,N-dimethylhydrazine (75 mL) was heated under reflux in an atmosphere of nitrogen for 30 hours. The solvent was removed in vacuo. The residue was treated with a mixture of water (200 mL), 1N HCl (75 mL), and ethyl acetate (100 mL). The organic extract was separated and the aqueous layer was extracted with more ethyl acetate (3×100 mL). The combined organic extracts were dried over Na₂SO₄, filtered, and concentrated under reduced pressure to give the product as white solid. ¹H NMR (400 MHz, d₆-DMSO) δ 13.2 (br s, 1H), 7.3-7.45 (m, 5H), 5.09 (s, 2H), 3.81 (s, 3H); ES MS (M+1)=277.

### Step 2: Methyl 4-(benzyloxy)-5-{[[(2R)-2-hydroxy-2-phenylethyl](methyl)amino]carbonyl}-1H-pyrazole-3-carboxylate

A mixture of methyl 4-benzyloxy-3-carboxy-1H-pyrazole-5-carboxylate (1.5 g, 5.43 mmol), HOAT (1.11 g, 8.15 mmol), (1R)-2-(methylamino)-1-phenylethanol (1.23 g, 8.15 mmol; prepared as described in Gurjar, M. K., et al., Org. Process Res. Dev. 1998, 2, 422.), triethylamine (0.82 g, 8.15 mmol), and EDC (1.56 g, 8.15 mmol) in DMF (20 mL) was stirred at room temperature for 3 hours. The product mixture was concentrated under vacuum and the residue was partitioned between saturated aqueous NH₄Cl and EtOAc. The layers were separated and the aqueous was extracted twice more with EtOAc. The combined organic extracts were dried over Na₂SO₄, filter, and concentrated in vacuo to afford the title product. ES MS (M+1)=410.

### Step 3: Methyl (7S)-3-(benzyloxy)-5-methyl-4-oxo-7-phenyl-4,5,6,7-tetrahydropyrazolo[1,5-a]pyrazine-2-carboxylate

To a solution of methyl 4-(benzyloxy)-5-{[[(2R)-2-hydroxy-2-phenylethyl](methyl)amino]carbonyl}-1H-pyrazole-3-carboxylate (1.84 g, 4.49 mmol) and triphenylphosphine (4.12 g, 15.7 mmol) in THF (20 mL) at room temperature, diethyl azodicarboxylate (2.85 mL, 15.7 mmol) was added dropwise over a period of 5 minutes. The mixture was stirred for 2 hours. The reaction mixture was concentrated under vacuum and the residue was purified by preparative HPLC (Waters prep LC 4000 System using a Waters Nova Pak column (3 100×40 mm I.D. cartridges, C18, 6 μM pore size) eluting with 95-5% water (0.10% TFA)/acetonitrile (0.10% TFA) at 60 mL/minute). Collection and concentration of appropriate fractions provided the title compound as yellow solid. ¹H NMR (400 MHz, d₆-DMSO) δ 7.49 (m, 2H), 7.3-7.4 (m, 6H), 7.02 (m, 2H), 5.81 (t, J=4.4 Hz, 1H), 5.27 (AB quartet, J=10.9 Hz, 2H), 4.19 (dd, J=13.5, 4.7 Hz, 1H), 3.88 (dd, J=13.5, 4.5 Hz, 1H), 3.77 (s, 3H), 2.93 (s, 3H); ES MS (M+1)=392.

### Step 4: (7S)-3-(Benzyloxy)-5-methyl-7-phenyl-4,5,6,7-tetrahydropyrazolo[1,5-a]pyrazine-2-carboxylic acid

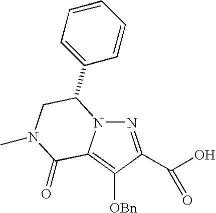

A mixture of methyl (7S)-3-(benzyloxy)-5-methyl-4-oxo-7-phenyl-4,5,6,7-tetrahydropyrazolo[1,5-a]pyrazine-2-carboxylate (0.74 g, 1.89 mmol) and 1N NaOH (3.79 mL, 3.79 mmol) in methanol (8 mL) was stirred at room temperature overnight. The mixture was acidified to pH 4 with 1N HCl and concentrated under vacuum. The residue was partitioned between water and ethyl acetate. The layers were then separated and the aqueous layer was extracted with more ethyl acetate (2×10 mL). The combined organic extracts were dried over Na₂SO₄, filtered, and concentrated under vacuum to give the title product as white foam. ES MS (M+1)=378.

### Step 5: (7S)-3-(Benzyloxy)-N-[(4-fluorophenyl)acetyl]-5-methyl-7-phenyl-4,5,6,7-tetrahydropyrazolo[1,5-a]pyrazine-2-carbohydrazide

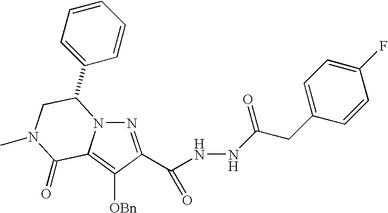

A solution of (7S)-3-(benzyloxy)-5-methyl-7-phenyl-4,5,6,7-tetrahydropyrazolo[1,5-a]pyrazine-2-carboxylic acid (148 mg, 0.39 mmol), 2-(4-fluorophenyl)acetohydrazide (99 mg, 0.59 mmol; prepared from 4-fluorophenyl acetyl chloride and hydrazine in a manner similar to that described in J. Heterocyclic Chemistry 1977, 14, 1123), EDC (113 mg, 0.59 mmol), HOBT (79 mg, 0.59 mmol) and triethylamine (82 μL, 0.59 mmol) in DMF (1 mL) was stirred at room temperature for 1 hour. The DMF was removed in vacuo and the residue was partitioned between water and ethyl acetate, adjusting to a pH of 3 using 1N HCl. The layers were then separated and the aqueous layer was extracted with more ethyl acetate (2×10 mL). The combined organic extracts were dried over Na₂SO₄, filtered, and concentrated under reduced pressure to give the product as white solid. ¹H NMR (400 MHz, d₆-DMSO) δ 10.2 (s, 1H), 9.84 (s, 1H), 7.98 (dd, J=8.4 Hz, 1H), 7.72 (d, J=8.4 Hz, 1H), 7.56-7.51 (m, 2H), 7.43-7.34 (m, 6H), 7.14 (t, J=8.9 Hz, 2H), 6.97 (d, J=6.1 Hz, 2H), 5.79 (m, 1H), 5.34 (AB quartet, J=10.9 Hz, 2H), 4.23 (dd, J=13, 4.7 Hz, 1H), 3.84 (dd, J=13.7, 3.7 Hz, 1H), 3.52 (s, 2H), and 2.92 (s, 3H). ES MS (M+1)=528.

### Step 6: (7S)-3-(Benzyloxy)-2-[5-(4-fluorobenzyl)-1,3,4-thiadiazol-2-yl]-5-methyl-7-phenyl-6,7-dihydropyrazolo[1,5-a]pyrazin-4(5H)-one

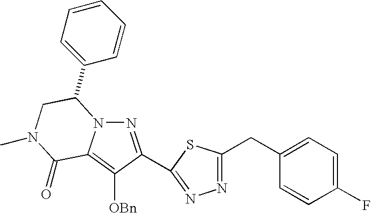

A mixture of (7S)-3-(benzyloxy)-N′-[(4-fluorophenyl)acetyl]-5-methyl-7-phenyl-4,5,6,7-tetrahydropyrazolo[1,5-a]pyrazine-2-carbohydrazide (90 m g, 0.16 mmol) and Lawesson's reagent (153 mg, 0.38 mmol) in anhydrous THF (1 μL) was heated under reflux for 2 hours. The product mixture was concentrated under vacuum. The solid residue was subjected to high pressure column chromatography purification with C-18 stationary phase eluting with a gradient of 95-5% H₂O (0.1% TFA)/CH₃CN (0.1% TFA). Collection and concentration of the appropriate fractions afforded the title compound. ES MS (M+1)=526.

### Step 7: (7S)-2-[5-(4-Fluorobenzyl)-1,3,4-thiadiazol-2-yl]-3-hydroxy-5-methyl-7-phenyl-6,7-dihydropyrazolo[1,5-a]pyrazin-4(5H)-one

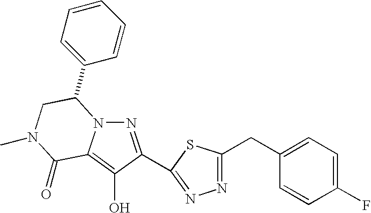

A mixture of (7S)-3-(benzyloxy)-2-[5-(4-fluorobenzyl)-1,3,4-thiadiazol-2-yl]-5-methyl-7-phenyl-6,7-dihydropyrazolo[1,5-a]pyrazin-4(5H)-one (0.069 g, 0.11 mmol) and HBr in acetic acid (1.5 mL, 33% solution in acetic acid) was stirred at room temperature overnight. The mixture was concentrated under reduced pressure. The residue was purified with high pressure column chromatography on a C-18 column using a gradient elution of 95-5% H₂O (0.1% TFA)/CH₃CN (0.1% TFA). Collection and concentration of the appropriate fractions afforded the title compound as light pink solid. ¹H NMR (400 MHz, d6-DMSO) δ 9.95 (br s, 1H), 7.43-7.32 (m, 5H), 7.18 (t, J=8.8 Hz, 2H), 7.08 (d, J=7.1 Hz, 2H), 5.78 (t, J=4.4 Hz, 1H), 4.48 (s, 2H), 4.18 (dd, J=13.4, 4.5 Hz, 1H), 3.86 (dd, J=13.4, 4.5 Hz, 1H), 2.91 (s, 3H); ES MS (M+1)=436.

### Example 5

### (7S)-2-[5-(4-Fluorobenzyl)-1,3,4-oxadiazol-2-yl]-3-hydroxy-5-methyl-7-phenyl-6,7-dihydropyrazolo[1,5a]pyrazin-4(5H)-one

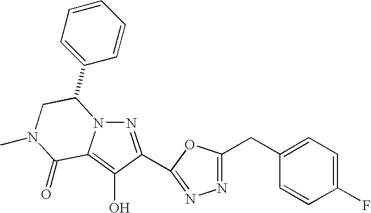

### Step 1: (7S)-3-(Benzyloxy)-2-[5-(4-fluorobenzyl)-1,3,4-oxadiazol-2-yl]-5-methyl-7-phenyl-6,7-dihydropyrazolo[1,5-a]pyrazin-4(5H)-one

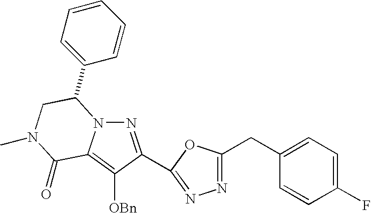

The title compound was prepared from (7S)-3-(benzyloxy)-N′-[(4-fluorophenyl)acetyl]-5-methyl-7-phenyl-4,5,6,7-tetrahydropyrazolo[1,5-a]pyrazine-2-carbohydrazide (Example 4, Step 5) using a procedure similar to that found in Example 4, Step 6, except that the mixture was treated Burgess Reagent instead of Lawesson's Reagent to afford the title compound as a white solid. ES MS (M+1)=510.

### Step 2: (7S)-2-[5-(4-Fluorobenzyl)-1,3,4-oxadiazol-2-yl]-3-hydroxy-5-methyl-7-phenyl-6,7-dihydropyrazolo[1,5-a]pyrazin-4(5H)-one

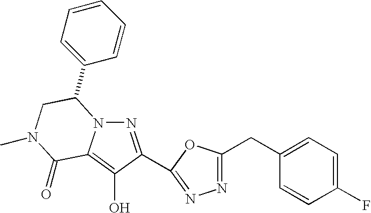

A mixture of (7S)-3-(benzyloxy)-2-[5-(4-fluorobenzyl)-1,3,4-oxadiazol-2-yl]-5-methyl-7-phenyl-6,7-dihydropyrazolo[1,5-a]pyrazin-4(5H)-one (52 mg, 0.102 mmol) and 10% Pd on carbon (5 mg) in MeOH (2 mL) was stirred under an atmosphere of hydrogen gas at room temperature for two hours. The reaction mixture was filtered through a pad of Celite, and the filter cake was washed with additional MeOH. The resulting filtrate was concentrated under vacuum to afford the title compound as light pink solid. ¹H NMR (400 MHz, d6-DMSO) δ 9.75 (br s, 1H), 7.40-7.32 (m, 5H), 7.17 (t, J=8.8 Hz, 2H), 7.09 (d, J=6.5 Hz, 2H), 5.78 (t, J=4.5 Hz, 1H), 4.32 (s, 2H), 4.16 (dd, J=13.5, 4.6 Hz, 1H), 3.87 (dd, J=13.4, 4.8 Hz, 1H), and 2.91 (s, 3H). ES MS (M+1)=420.

### Example 6

### HIV Integrase Assay

### Strand Transfer Catalyzed by Recombinant Integrase

Assays for the strand transfer activity of integrase were conducted in accordance with WO 02/30930 for recombinant integrase. The compounds prepared in Examples 1-5 were tested in this integrase assay and all were found to have IC₅₀ values of less than or equal to 1 micromolar.

Further description on conducting the assay using preassembled complexes is found in Wolfe, A. L. et al., J. Virol. 1996, 70: 1424-1432, Hazuda et al., J. Virol. 1997, 71: 7005-7011; Hazuda et al., Drug Design and Discovery 1997, 15: 17-24; and Hazuda et al., Science 2000, 287: 646-650.

### Example 7

### Assays for Inhibition of HIV Replication

An assay for measuring the inhibition of acute HIV infection with HeLa P4-2 cells in a single cycle infectivity assay (alternatively referred to as the “vertical” assay) was conducted in accordance with Joyce, J. G., et al., J. Biol. Chem., 2002, 277, 45811, Hazuda, D. J. et al., Science, 2000, 287, 646, and Kimpton, J. et al, J. Virol. 1992, 66, 2232. The compounds of Examples 1 to 5 were found to have IC₅₀ values of less than 35 micromolar in the vertical assay.

While the foregoing specification teaches the principles of the present invention, with examples provided for the purpose of illustration, the practice of the invention encompasses all of the usual variations, adaptations and/or modifications that come within the scope of the following claims.

1. A compound of Formula (I) or a pharmaceutically acceptable salt thereof: 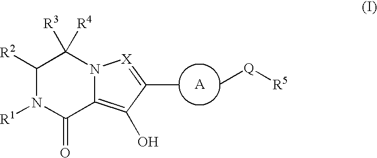
wherein
R¹ is —H, —C₁₋₆ alkyl, —C₃₋₆ cycloalkyl, or —C₁₋₆ alkyl which is substituted with 1 or 2 substituents each of which is independently:
(1) C₃₋₈ cycloalkyl,
(2) aryl,
(3) a 5- or 6-membered saturated or mono-unsaturated heterocyclic ring containing from 1 to 4 heteroatoms independently selected from N, O and S,
(4) a 5- or 6-membered heteroaromatic ring containing from 1 to 4 heteroatoms independently selected from N, O and S, or
(5) a 9- or 10-membered fused bicyclic heterocycle containing from 1 to 4 heteroatoms independently selected from N, O and S, wherein at least one of the rings is aromatic;
wherein
(A) each cycloalkyl is optionally substituted with from 1 to 3 substituents, each of which is independently halo, —C₁₋₆ alkyl, —O—C₁₋₆ alkyl, —OH, or C₁₋₆ haloalkyl;
(B) each aryl is optionally substituted with from 1 to 5 substituents each of which is independently
(1) —C₁₋₆ alkyl, optionally substituted with from 1 to 3 substituents each of which is independently —OH, —O—C₁₋₆ alkyl, —O—C₁₋₆ haloalkyl, —CN, —NO₂, —N(R^{a}R^{b}), —C(═O)N(R^{a}R^{b}), —C(═O)R^{a}, —CO₂R^{c}, —SH, —S(O)ₙR^{d}, —SO₂N(R^{a}R^{b}), —N(R^{a})C(═O)R^{b}, —N(R^{a})CO₂R^{d}, —N(R^{a})SO₂R^{d}, —N(R^{a})SO₂N(R^{a}R^{b}), —OC(═O)N(R^{a}R^{b}), or —N(R^{a})C(═O)N(R^{a}R^{b}),
(2) —O—C₁₋₆ alkyl, optionally substituted with from 1 to 3 substituents each of which is independently —OH, —O—C₁₋₆ alkyl, —O—C₁₋₆ haloalkyl, —SH, —S(O)ₙR^{d}, —C(═O)N(R^{a}R^{b}), —SO₂N(R^{a}R^{b}), —N(R^{a})C(═O)R^{b}, —N(R^{a})CO₂R^{d}, —N(R^{a})SO₂R^{d}, —N(R^{a})SO₂N(R^{a}R^{b}), —OC(═O)N(R^{a}R^{b}), or —N(R^{a})C(═O)N(R^{a}R^{b}),
(3) —C₁₋₆ haloalkyl,
(4) —O—C₁₋₆ haloalkyl,
(5) —OH,
(6) halo,
(7) —CN,
(8) —NO₂,
(9) —N(R^{a}R^{b}),
(10) —C(═O)N(R^{a}R^{b}),
(11) —C(═O)R^{a},
(12) —CO₂R^{c},
(13) —SR^{c},
(14) —S(═O)R^{d},
(15) —SO₂R^{d},
(16) —N(R^{a})SO₂R^{d},
(17) —SO₂N(R^{a}R^{b}),
(18) —N(R^{a})C(═O)R^{b}, or
(19) —N(R^{a})CO₂R^{d};
(C) each saturated or mono-unsaturated heterocyclic ring is
(i) optionally substituted with from 1 to 5 substituents each of which is independently halogen, —C₁₋₆ alkyl, —C₁₋₆ haloalkyl, —O—C₁₋₆ alkyl, —O—C₁₋₆ haloalkyl, or oxo; and
(ii) optionally substituted with 1 or 2 substituents each of which is independently aryl or a 5- or 6-membered heteroaromatic ring containing from 1 to 4 heteroatoms independently selected from N, O and S; and
(D) each heteroaromatic ring or each fused bicyclic heterocycle is
(i) optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, or oxo; and
(ii) optionally substituted with 1 or 2 substituents each of which is independently aryl or —C₁₋₆ alkyl-aryl;
R² is —H or —C₁₋₆ alkyl;
R³ is —H, —C₁₋₆ alkyl, —C₁₋₆ haloalkyl, or —C₁₋₆ alkyl substituted with one of —OH, —O—C₁₋₆ alkyl, —O—C₁₋₆ haloalkyl, —CN, —NO₂, —N(R^{a}R^{b}), —C(═O)N(R^{a}R^{b}), —C(═O)R^{a}, —CO₂R^{c}, —SH, —S(O)ₙR^{d}, —SO₂N(R^{a}R^{b}), —N(R^{a})C(═O)R^{b}, —N(R^{a})CO₂R^{d}, —N(R^{a})SO₂R^{d}, —N(R^{a})SO₂N(R^{a}R^{b}), —OC(═O)N(R^{a}R^{b}), or —N(R^{a})C(═O)N(R^{a}R^{b});
R⁴ is:
(1) —H,
(2) —C₁₋₆ alkyl optionally substituted with one of —OH, —O—C₁₋₆ alkyl, —O—C₁₋₆ haloalkyl, —CN, —NO₂, —N(R^{a}R^{b}), —C(═O)N(R^{a}R^{b}), —C(═O)R^{a}, —CO₂R^{c}, —SH, —S(O)ₙR^{d}, —SO₂N(R^{a}R^{b}), —N(R^{a})—C(R^{b})═O, —N(R^{a})SO₂R^{d}, —N(R^{a})SO₂N(R^{a}R^{b}), —OC(═O)N(R^{a}R^{b}), —N(R^{a})C(═O)N(R^{a}R^{b}), —O—C₁₋₆ alkyl-C(═O)N(R^{a}R^{b}), —S—C₁₋₆ alkyl-C(═O)N(R^{a}R^{b}), —N(R^{a})—C₁₋₆ alkyl-C(═O)N(R^{a}R^{b}), or —N(SO₂R^{d})—C₁₋₆ alkyl-C(═O)N(R^{a}R^{b}),
(3) —C₁₋₆ haloalkyl,
(4) —C(═O)R^{a},
(5) —CO₂R^{c},
(6) —C(═O)N(R^{a}R^{b}),
(7) —SO₂N(R^{a}R^{b}),
(8) —C₂₋₆ alkenyl,
(9) —C₂₋₆ alkenyl-C(═O)—N(R^{a})₂,
(10) —C₂₋₅ alkynyl,
(11) —C₂₋₅ alkynyl-CH₂N(R^{a})₂,
(12) —C₂₋₅ alkynyl-CH₂OR^{a},
(13) —C₂₋₅ alkynyl-CH₂S(O)ₙR^{c}, or
(14) —R^{j},
(15) —C₁₋₆ alkyl substituted with R^{j},
(16) —C₁₋₆ haloalkyl substituted with R^{j},
(17) —C₁₋₆ alkyl-O—R^{j},
(18) —C₁₋₆ alkyl-O—C₁₋₆ alkyl-R^{j},
(19) —C₁₋₆ alkyl-S(O)ₙ—R^{j},
(20) —C₁₋₆ alkyl-S(O)ₙ—C₁₋₆ alkyl-R^{j},
(21) —C₁₋₆ alkyl-N(R^{a})—R^{j},
(22) —C₁₋₆ alkyl-N(R^{a})—C₁₋₆ alkyl-R^{j},
(23) —C₁₋₆ alkyl-N(R^{a})—C₁₋₆ alkyl-OR^{j}, with the proviso that the —N(R^{a})— moiety and the —OR^{j} moiety are not both attached to the same carbon of the —C₁₋₆ alkyl-moiety,
(24) —C₁₋₆ alkyl-C(═O)—R^{j},
(25) —C₁₋₆ alkyl-C(═O)N(R^{a})—R^{j},
(26) —C₁₋₆ alkyl-N(R^{a})C(═O)—R^{j},
(27) —C₁₋₆ alkyl-C(═O)N(R^{a})—C₁₋₆ alkyl-R^{j}, or,
(28) —C₁₋₆ alkyl-N(R^{a})—C₀₋₆ alkyl-S(O)ₙRⁱ;
wherein R^{j} is
(i) aryl, which is optionally substituted with from 1 to 5 substituents each of which is independently —C₁₋₆ alkyl, —C₁₋₆ alkyl-OH, —C₁₋₆ alkyl-O—C₁₋₆ alkyl, —C₁₋₆ alkyl-O—C₁₋₆ haloalkyl, —C₁₋₆ alkyl-N(R^{a}R^{b}), —C₁₋₆ alkyl-C(═O)N(R^{a}R^{b}), —C₁₋₆ alkyl-C(═O)R^{a}, —C₁₋₆ alkyl-CO₂R^{c}, —C₁₋₆ alkyl-S(O)ₙR^{c}, —O—C₁₋₆ alkyl, —C₁₋₆ haloalkyl, —O—C₁₋₆ haloalkyl, —OH, halo, —N(R^{a}R^{b}), —C(═O)N(R^{a}R^{b}), —C(═O)R^{a}, —CO₂R^{c}, —SH, —S(O)ₙR^{d}, or —SO₂N(R^{a}R^{b});
(ii) a 4- to 7-membered saturated or mono-unsaturated heterocyclic ring containing at least one carbon atom and from 1 to 4 heteroatoms independently selected from N, O and S, wherein the heterocyclic ring is:
(a) optionally substituted with from 1 to 5 substituents each of which is independently halogen, —C₁₋₆ alkyl, —C₁₋₆ haloalkyl, —O—C₁₋₆ alkyl, —O—C₁₋₆ haloalkyl, or oxo; and
(b) optionally mono-substituted with aryl or HetA;
wherein HetA is a 5- or 6-membered heteroaromatic ring containing from 1 to 4 heteroatoms independently selected from N, O and S, wherein the heteroaromatic ring is optionally fused with a benzene ring, and HetA is optionally substituted with from 1 to 4 substituents each of which is independently —C₁₋₆ alkyl, —C₁₋₆ haloalkyl, —O—C₁₋₆ alkyl, —O—C₁₋₆ haloalkyl, or oxo; or
(iii) a 5- or 6-membered heteroaromatic ring containing from 1 to 4 heteroatoms independently selected from N, O and S, wherein the heteroaromatic ring is optionally substituted with from optionally substituted with from 1 to 4 substituents each of which is independently —C₁₋₆ alkyl, —C₁₋₆ haloalkyl, —O—C₁₋₆ alkyl, —O—C₁₋₆ haloalkyl, or oxo;
ring A is a 5- or 6-membered saturated, partially saturated, or aromatic monocyclic ring or a 8- to 11-membered saturated, partially saturated, or aromatic bicyclic ring, wherein said monocyclic or bicyclic ring contains from 1 to 4 heteroatoms independently selected from N, O and S;
Q is C₁₋₆ alkylene, —NR⁶—, —O—, —C(O)—, —CH(OR⁶)—, —S(O)₂—, or —CF₂—;
R⁵ is
(1) C₃₋₈ cycloalkyl wherein said cycloalkyl is optionally substituted with aryl and said cycloalkyl is optionally substituted with from 1 to 3 substituents, each of which is independently halo, —C₁₋₆ alkyl, —O—C₁₋₆ alkyl, —OH, or C₁₋₆ haloalkyl,
(2) aryl,
(3) a fused bicyclic carbocycle consisting of a benzene ring fused to a C₅₋₇ cycloalkyl,
(4) a 5- or 6-membered saturated or partially saturated heterocyclic ring containing from 1 to 4 heteroatoms independently selected from N, O and S,
(5) a 5- or 6-membered heteroaromatic ring containing from 1 to 4 heteroatoms independently selected from N, O and S, or
(6) a 9- or 10-membered fused bicyclic heterocyclic ring containing from 1 to 4 heteroatoms independently selected from N, O and S, wherein at least one of the rings is aromatic;
wherein
each aryl in (1) or (2) or each fused carbocycle in (3) is optionally substituted with one or more substituents (e.g., optionally from 1 to 5, or 1 to 4, or 1 to 3, or 1 or 2 substituents; or is optionally mono-substituted) each of which is independently halogen, —OH, —C₁₋₆ alkyl, —C₁₋₆ alkyl-OR^{a}, —C₁₋₆ haloalkyl, —O—C₁₋₆ alkyl, —O—C₁₋₆ haloalkyl, —CN, —NO₂, —N(R^{a}R^{b}), —C₁₋₆ alkyl-N(R^{a}R^{b}), —C(═O)N(R^{a}R^{b}), —C(═O)R^{a}, —CO₂R^{a}, —C₁₋₆ alkyl-CO₂R^{a}, —OCO₂R^{a}, —SR^{a}, —S(═O)R^{a}, —SO₂R^{a}, —N(R^{a})SO₂R^{b}, —SO₂N(R^{a}R^{b}), —N(R^{a})C(═O)R^{b}, —N(R^{a})CO₂R^{b}, —C₁₋₆ alkyl-N(R^{a})CO₂R^{b}, aryl, —C₁₋₆ alkyl-aryl, —O-aryl, or —C₀₋₆ alkyl-het wherein het is a 5- or 6-membered heteroaromatic ring containing from 1 to 4 heteroatoms independently selected from N, O and S, and het is optionally fused with a benzene ring, and is optionally substituted with one or more substituents (e.g., optionally from 1 to 5, or 1 to 4, or 1 to 3, or 1 or 2 substituents; or is optionally mono-substituted) each of which is independently —C₁₋₆ alkyl, —C₁₋₆ haloalkyl, —O—C₁₋₆ alkyl, —O—C₁₋₆ haloalkyl, oxo, or —CO₂R^{a};
each saturated or unsaturated non-aromatic heterocyclic ring in (4) is optionally substituted with one or more substituents (e.g., optionally from 1 to 6, or 1 to 5, or 1 to 4, or 1 to 3, or 1 or 2 substituents; or is optionally mono-substituted) each of which is independently halogen, —C₁₋₆ alkyl, —C₁₋₆ haloalkyl, —O—C₁₋₆ alkyl, —O—C₁₋₆ haloalkyl, oxo, aryl, or a 5- or 6-membered heteroaromatic ring containing from 1 to 4 heteroatoms independently selected from N, O and S; and
each heteroaromatic ring in (5) or each fused bicyclic heterocycle in (6) is optionally substituted with one or more substituents (e.g., optionally from 1 to 6, or 1 to 5, or 1 to 4, or 1 to 3, or 1 or 2 substituents; or is optionally mono-substituted) each of which is independently halogen, —C₁₋₆ alkyl, —C₁₋₆ haloalkyl, —O—C₁₋₆ alkyl, —O—C₁₋₆ haloalkyl, oxo, aryl, or —C₁₋₆ alkyl-aryl;
R⁶ is —H, —C₁₋₆ alkyl, C₃₋₈ cycloalkyl, —C₁₋₆ haloalkyl, aryl, ar(C₁₋₃)alkyl, or HetB;
HetB is a 3- or 7-membered saturated, partially saturated, or aromatic monocyclic ring or a 8- to 11-membered saturated, partially saturated, or aromatic bicyclic ring, wherein said monocyclic or bicyclic ring contains from 1 to 4 heteroatoms independently selected from N, O and S;
each R^{a}, R^{b}, and R^{c} is independently —H or —C₁₋₆ alkyl;
each n is independently an integer equal to zero, 1 or 2;
X is N or C(R⁷);
R⁷ is —H or —C₁₋₆ alkyl; and
each R^{d} is independently —C₁₋₆ alkyl.

2. The compound of claim 1, wherein the compound is of Formula (I-1), 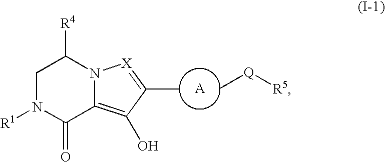
wherein R¹ is —C₁₋₃ alkyl; and X is N or CH.

3. The compound of claim 2, wherein R⁴ is H or —R^{j}; and R^{j} is:
(i) phenyl, which is optionally substituted with from 1 to 3 substituents each of which is independently —C₁₋₄ alkyl, —C₁₋₄ alkyl-OH, —C₁₋₄ alkyl-O—C₁₋₄ alkyl, —C₁₋₄ alkyl-O—C₁₋₄ haloalkyl, —C₁₋₄ alkyl-N(R^{a}R^{b}), —C₁₋₄ alkyl-C(═O)N(R^{a}R^{b}), —C₁₋₄ alkyl-C(═O)R^{a}, —C₁₋₄ alkyl-CO₂R^{c}, —C₁₋₄ alkyl-S(O)ₙR^{d}, —O—C₁₋₄ alkyl, —C₁₋₄ haloalkyl, —O—C₁₋₄ haloalkyl, —OH, halo, —N(R^{a}R^{b}), —C(═O)N(R^{a}R^{b}), —C(═O)R^{a}, —CO₂R^{c}, —SH, —S(O)ₙR^{d}, or —SO₂N(R^{a}R^{b}); or
(ii) a saturated heterocyclic ring selected from the group consisting of piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, thiadiazepanyl, dithiazepanyl, azepanyl, diazepanyl, thiadiazinanyl, and dioxanyl; wherein the saturated heterocyclic ring is:
(a) optionally substituted with from 1 to 4 substituents each of which is independently halogen, —C₁₋₄ alkyl, —C₁₋₄ haloalkyl, —O—C₁₋₄ alkyl, —O—C₁₋₄ haloalkyl, or oxo; and
(b) optionally mono-substituted with phenyl or HetA; wherein HetA is a heteroaromatic ring selected from the group consisting of pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl; wherein the heteroaromatic ring is optionally substituted with from 1 to 3 substituents each of which is independently —C₁₋₄ alkyl, —C₁₋₄ haloalkyl, —O—C₁₋₄ alkyl, —O—C₁₋₄ haloalkyl, or oxo.

4. The compound of claim 3, wherein R^{j} is phenyl, which is optionally substituted with from 1 to 3 substituents each of which is independently —C₁₋₄ alkyl, —C₁₋₄ alkyl-OH, —C₁₋₄ alkyl-O—C₁₋₄ alkyl, —C₁₋₄ alkyl-O—C₁₋₄ haloalkyl, —C₁₋₄ alkyl-N(R^{a}R^{b}), —C₁₋₄ alkyl-C(═O)N(R^{a}R^{b}), —C₁₋₄ alkyl-C(═O)R^{a}, —C₁₋₄ alkyl-CO₂R^{d}, —C₁₋₄ alkyl-S(O)ₙR^{d}, —O—C₁₋₄ alkyl, —C₁₋₄ haloalkyl, —O—C₁₋₄ haloalkyl, —OH, halo, —N(R^{a}R^{b}), —C(═O)N(R^{a}R^{b}), —C(═O)R^{a}, —CO₂R^{c}, —SH, —S(O)ₙR^{d}, or —SO₂N(R^{a}R^{b}).

5. The compound of claim 4, wherein ring A is a 5- or 6-membered aromatic monocyclic ring containing from 1 to 4 heteroatoms independently selected from N, O and S.

6. The compound of claim 5, wherein ring A is oxadiazolyl, triazolyl, thiadazolyl, oxazolyl, tetrazolyl or pyrimidinyl.

7. The compound of claim 6, wherein ring A is 1,2,4-oxadiazolyl, 1,3,4-oxadiazolyl, 1,2,4-triazolyl, 1,3,4-thiadazolyl, 1,3-oxazol-2-yl, 2-tetrazol-5-yl, 1-tetrazol-5-yl or 4-pyrimidinyl.

8. The compound of claim 5, wherein Q is —C₁₋₃ alkylene.

9. The compound of claim 8, wherein Q is —CH₂—.

10. The compound of claim 9, wherein R⁵ is phenyl, wherein the phenyl is optionally substituted with from 1 to 3 substituents each of which is independently selected from fluoro, bromo, chloro, —OH, —C₁₋₄ alkyl, —C₁₋₄ fluoroalkyl, —O—C₁₋₄ alkyl, —O—C₁₋₄ fluoroalkyl, —(CH₂)₁₋₂—N(R^{a}R^{b}), —SO₂R^{a}, —(CH₂)₀₋₂—CO₂R^{a}, —(CH₂)₀₋₂—N(R^{a})CO₂R^{b}, —NO₂, —SR^{a}, —N(R^{a}R^{b}) and phenyl.

11. The compound of claim 1, wherein the compound is:

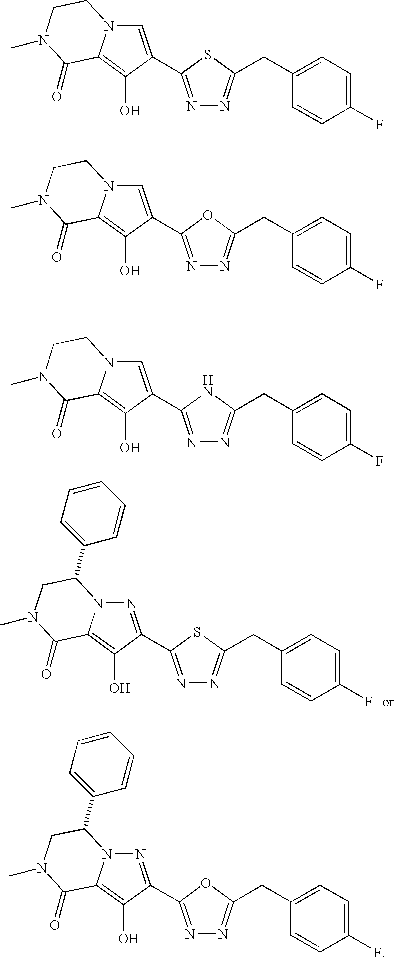

12. A pharmaceutical composition comprising an effective amount of a compound or a pharmaceutically acceptable salt thereof according to claim 1, and a pharmaceutically acceptable carrier.

13. (canceled)

14. A method for treating infection by HIV or for treating or delaying the onset of AIDS in a subject in need thereof which comprises administering to the subject an effective amount of the compound or a pharmaceutically acceptable salt thereof according to claim 1.

15. (canceled)

16. (canceled)

17. (canceled)

18. (canceled)

19. A combination which is (i) a compound or a pharmaceutically acceptable salt thereof according to claim 1, and (ii) an HIV infection/AIDS antiviral agent selected from the group consisting of HIV protease inhibitors, non-nucleoside HIV reverse transcriptase inhibitors and nucleoside HIV reverse transcriptase inhibitors; wherein the compound of (i) or its pharmaceutically acceptable salt and the HIV infection/AIDS antiviral agent of (ii) are each employed in an amount that renders the combination effective for treating infection by HIV, or for treating or delaying the onset of AIDS.
